(19) United States
(12) Patent Application Publication
(10) Pub. No.: US 2011/0129822 A1
(43) Pub. Date: Jun. 2, 2011

(54) MULTI DRUG RESPONSE MARKERS FOR BREAST CANCER CELLS

(75) Inventors: Kui SHEN, Pittsburgh, PA (US); Nan Song, Pittsburgh, PA (US); Shara D. Rice, Pittsburgh, PA (US); Dakun Wang, Pittsburgh, PA (US); David A. Gingrich, Pittsburgh, PA (US); Zhenyu Ding, Pittsburgh, PA (US); Chunqiao Tian, Pittsburgh, PA (US); Stacey L. Brower, Pittsburgh, PA (US); Paul R. Ervin, Pittsburgh, PA (US); Michael Gabrin, Pittsburgh, PA (US)

(73) Assignee: Precision Therapeutics, Inc., Pittsburgh, PA (US)

(21) Appl. No.: 12/957,604

(22) Filed: Dec. 1, 2010

Related U.S. Application Data
(60) Provisional application No. 61/265,588, filed on Dec. 1, 2009.
(60) Provisional application No. 61/364,446, filed on Jul. 15, 2010.

(51) Int. Cl. C12Q 1/68

(52) U.S. Cl. 435/6.1

(57) ABSTRACT

The present invention provides methods for preparing a gene expression profile of a breast cancer cell, tumor, or cell line, where the gene expression profile may be evaluated for one or more gene expression signatures indicative of multidrug resistance. The signature may be indicative of resistance to one or more chemotherapeutic agents selected from a Taxol (e.g., Docetaxel or Paclitaxel), an antibiotic (e.g., Doxorubicin or Epirubicin), an antimetabolite (e.g., Fluorouracil and/or Gemcitabine), and an alkylating agent (e.g., Cyclophosphamide). Generally, the gene expression profile contains the level of expression for a plurality of genes listed in FIGS. 3, 4, and/or 5. Gene expression profiles for evaluating multidrug resistance for ER positive and ER negative breast cancers are also provided.

### PRIORITY

This provisional application claims priority to U.S. Provisional Application No. 61/265,588 filed Dec. 1, 2009, and U.S. Provisional Application No. 61/364,446 filed Jul. 15, 2010, which are both hereby incorporated by reference in their entireties.

### BACKGROUND

A major obstacle in the effective treatment of breast cancer with chemotherapeutic agents is the phenomenon of multidrug resistance. Standards of care have involved various neoadjuvant approaches to chemotherapy and surgical resection, with the greatest success occurring when tumor tissue is surgically removed and patients are subsequently treated with chemotherapy. Generally, the success rate is less than 50% with primary breast cancer, and chemotherapeutic agents are less effective in treating recurrent disease due to drug resistance. In fact, resistant patients tend to be resistant to multiple drugs despite their different cytotoxic mechanisms.

Understanding the molecular mechanisms of multidrug resistance has important biological significance and potential clinical, diagnostic, and prognostic utility, for example, by facilitating drug selection studies, identifying new therapeutic targets, in addition to individualizing patient therapy.

Recent advances in genomic technology have provided an opportunity to identify genes associated with cancer drug resistance. Studies using tumor tissue from breast cancer patients have identified gene expression profiles potentially associated with clinical outcome (van de Vijver, He et al. 2002; Chang, Wooten et al. 2003; Gianni, Zambetti et aI. 2005; Iwao-Koizumi, Matoba et aI. 2005; Wang, Klijn et al. 2005; Hess, Anderson et al. 2006; Paik, Tang et al. 2006; Liedtke, Hatzis et al. 2009). However, in identifying gene expression profiles with clinical or biological significance, use of patient tumor tissue can be disadvantageous, due to the limited source of tissue, the long time necessary to assess clinical outcome, and the fact that each patient can be initially treated with only one panel of drugs. To overcome these problems, cell lines may be used as a proxy for patient tumor tissue using chemosensitivity and resistance assays (CSRA) (Staunton, Slonim et al. 2001; Dan, Tsunoda et al. 2002; Mariadason, Arango et al. 2003; Kang, Kim et al. 2004); (Gyorffy, Surowiak et al. 2006). While gene expression profiles have been identified with some correlation to multi-drug response, most studies have used cell lines of heterogeneous origin, e.g., not exclusively breast cancer cell lines. Moreover, since breast cancer cell lines are very heterogeneous, including ER positive and ER negative cell lines, breast cancer cells may have several distinct response patterns to chemotherapeutic agents. That is, different cellular mechanisms may contribute to multidrug resistance.

Multidrug response gene expression profiles are needed to assess chemosenstivity/resistance in breast cancer cells, including in ER+ and ER− breast cancer cells.

### SUMMARY OF THE INVENTION

The present invention provides methods for preparing a gene expression profile of a breast cancer cell, tumor, or cell line, where the gene expression profile contains the expression level for genes indicative of multidrug responsiveness (sensitivity or resistance). The profile may be evaluated for the presence of one or more gene expression signatures indicative of responsiveness to one or more drugs. The gene expression signature may be indicative of sensitivity or resistance to one or more chemotherapeutic agents selected from a taxol (e.g., docetaxel or paclitaxel), an antibiotic (e.g., doxorubicin or epirubicin), an antimetabolite (e.g., fluorouracil and/or gemcitabine), and an alkylating agent (e.g., cyclophosphamide). The gene expression signature may be indicative of a multidrug resistant breast cancer cell.

Generally, the gene expression signatures described herein can be defined by the level of gene expression exhibited by drug-sensitive breast cancer cell lines (immortal cell lines), versus the level of gene expression exhibited by drug-resistant breast cancer cell lines (immortal cell lines). Drug-sensitive and drug-resistant cell lines are defined by their drug response in an in vitro chemosensitivity assay, as described more fully herein. A collection of publicly available breast cancer cell lines, and their relative sensitivity to a panel of chemotherapeutic agents is described herein (see FIG. 2).

In certain embodiments, the gene expression profile contains the level of expression for a plurality of genes listed in FIG. 5, and as described in detail herein. In certain embodiments, the profile contains the level of expression for DBI, TOP2A, and PMVK which are differentially expressed in both estrogen receptor (ER) positive and ER negative multidrug resistant breast cancer cell lines.

In certain embodiments, the ER status of the tumor is determined or is known, which can aid evaluation of the gene expression profile for a gene expression signature indicative of drug response (e.g., multidrug resistance).

For example, where the breast cancer, tumor, or cell line is ER positive, the gene expression profile is evaluated for the presence of a gene expression signature that is indicative of drug sensitivity or resistance for an Estrogen Receptor (ER) positive breast cancer cell. In such embodiments, the ER positive gene expression signatures may be defined by the level of gene expression exhibited by ER positive drug-sensitive breast cancer cell lines (immortal cell lines), versus the level of gene expression exhibited by ER positive drug-resistant breast cancer cell lines (immortal cell lines). For example, the ER positive gene expression profile may contain the level of expression for a plurality of genes listed in FIG. 3, as described in detail herein.

In other embodiments where the breast cancer, tumor, or cell line is ER negative, the gene expression profile is evaluated for the presence of a gene expression signature that is indicative of drug sensitivity or resistance for an ER negative breast cancer cell. In such embodiments, the gene expression signatures may be defined by the level of gene expression exhibited by ER negative drug-sensitive breast cancer cell lines (immortal cell lines), versus the level of gene expression exhibited by ER negative drug-resistant breast cancer cell lines (immortal). The ER negative gene expression profile may contain the level of expression for a plurality of genes listed in FIG. 4, as described in detail herein.

In other aspects, the invention provides methods for determining whether a breast tumor is sensitive or resistant to multiple drugs, such as a plurality of agents selected from taxol (e.g., docetaxel or paclitaxel), an antibiotic (e.g., doxorubicin or epirubicin), an antimetabolite (e.g., fluorouracil and/or gemcitabine), and an alkylating agent (e.g., cyclophosphamide). The method generally comprises determining the gene expression profiles described herein for the breast tumor or malignant cells thereof, and evaluating the profile for the presence or absence of a gene expression signature indicative of multidrug response (e.g. resistance). In some embodiments, the ER status is also determined or is known, and gene expression signatures specific to ER-positive and ER-negative breast cancer cells are described herein.

As exemplified herein, 27 well-studied breast cancer cell lines were used to identify genes that are related to multidrug resistance in ER− and ER+ breast cancer cell lines. An in vitro chemoresponse assay was used as a proxy of drug response to determine the sensitivity of these cell lines to seven chemotherapy agents commonly used to treat breast cancer patients. The drug response profile of the breast cancer cell lines demonstrated that the in vitro assay is a good proxy for drug response. Through pharmacogenomic analysis, the expression levels for 524 genes were identified as related to multidrug resistance for all breast cell lines (FIG. 5). Many of these genes are related to ER status, which is consistent with the fact that ER status is related to drug response. Furthermore, 32 genes were identified that are related to multidrug response in ER negative breast cancer cell lines (FIG. 4), and 188 genes were identified that are related to multidrug response in ER positive cell lines (FIG. 3). Only 3 genes are in-common in both profiles (DBI, PMVK and TOP2A). Thus, the present application discloses that different genes are associated with multidrug response in ER-positive and ER-negative breast cancer cells.

### DESCRIPTION OF THE FIGURES

FIG. 1 is a heatmap of drug response for 27 breast cancer cell lines as determined by the CHEMOFX assay. Darker boxes represent sensitivity. The bar across the top indicates ER status of the cell line in the column below. Black corresponds to ER positive and grey corresponds to ER negative.

FIG. 2 summarizes the chemosensitivity of 27 breast cancer cell lines to 7 different drugs, measured by CHEMOFX. Lower numbers indicate sensitivity.

FIG. 3 lists 188 genes whose expression level is associated with multidrug resistance in ER-positive breast cancer cell lines (FIG. 3A). FIG. 3 includes measures of fold change between sensitive and resistant cell lines (FIG. 3B).

FIG. 4 lists 32 genes whose expression level is associated with multidrug resistance in ER-negative breast cancer cell lines (FIG. 4A). FIG. 4 includes measures of fold change between sensitive and resistant cell lines (FIG. 4B).

FIG. 5 lists 524 genes whose expression level is associated with multidrug resistance in all breast cancer cell lines (FIG. 5A). FIG. 5 includes measures of fold change between sensitive and resistant cell lines (FIG. 5B).

### DETAILED DESCRIPTION OF THE INVENTION

The invention provides methods for preparing gene expression profiles for breast tumor specimens or cell lines, as well as methods for evaluating a breast cancer's sensitivity and/or resistance to one or more chemotherapeutic agents or combinations of agents. For example, the gene expression profile generated for a tumor specimen, or cultured cells derived therefrom, is evaluated for the presence of one or more indicative gene expression signatures. The gene expression signatures are indicative of response (sensitivity or resistance) to one or more chemotherapeutic agents as described herein. In this aspect, the invention may provide information to guide a physician in designing/administering an individualized chemotherapeutic regimen for a breast cancer patient.

The patient generally is a breast cancer patient, and the tumor is generally a solid tumor of epithelial origin. The tumor specimen may be obtained from the patient by surgery, or may be obtained by biopsy, such as a fine needle biopsy or other procedure prior to the selection/initiation of neoadjuvant therapy. In certain embodiments, the breast cancer is preoperative or post-operative breast cancer. In certain embodiments, the patient has not undergone treatment to remove the breast tumor, and therefore is a candidate for neoadjuvant therapy.

The breast cancer may be primary or recurrent, and may be of any type (as described above), stage (e.g., Stage I, II, III, or IV or an equivalent of other staging system), and/or histology. The patient may be of any age, sex, performance status, and/or extent and duration of remission.

In certain embodiments, the patient is a candidate for treatment with one or more of taxol (e.g., docetaxel or paclitaxel), doxorubicin, epirubicin, an antimetabolite (e.g., fluorouracil and/or gemcitabine), and an alkylating agent (e.g., cyclophosphamide).

The gene expression profile is determined for the tumor tissue or cell sample, such as a tumor sample removed from the patient by surgery or biopsy. The tumor sample may be “fresh,” in that it was removed from the patent within about five days of processing, and remains suitable or amenable to culture. In some embodiments, the tumor sample is not “fresh,” in that the sample is not suitable or amenable to culture. Tumor samples are generally not fresh after from 3 to 7 days (e.g., about five days) of removal from the patient. The sample may be frozen after removal from the patient, and preserved for later RNA isolation. The sample for RNA isolation may be a formalin-fixed paraffin-embedded (FFPE) tissue. In certain embodiments, the tissue sample is not suitable for growing out malignant cells in a monolayer culture.

In certain embodiments, the tissue specimen is a transcutaneous biopsy-sized specimen, and generally contains less than about 100 mg of tissue, or in certain embodiments, contains about 50 mg of tissue or less. The tumor specimen (or biopsy) may contain from about 20 mg to about 50 mgs of tissue, such as about 35 mg of tissue. The tissue may be obtained, for example, as one or more (e.g., 1, 2, 3, 4, or 5) core needle biopsies (e.g., using a 14-gauge needle or other suitable size).

In certain embodiments, the malignant cells are enriched or expanded in culture by forming a monolayer culture from tumor sample explants. For example, cohesive multicellular particulates (explants) are prepared from a patient's tissue sample (e.g., a biopsy sample or surgical specimen) using mechanical fragmentation. This mechanical fragmentation of the explant may take place in a medium substantially free of enzymes that are capable of digesting the explant. Some enzymatic digestion may take place in certain embodiments, such as for ovarian or colorectal tumors.

For example, where it is desirable to expand and/or enrich malignant cells in culture relative to non-malignant cells that reside in the tumor, the tissue sample is systematically minced using two sterile scalpels in a scissor-like motion, or mechanically equivalent manual or automated opposing incisor blades. This cross-cutting motion creates smooth cut edges on the resulting tissue multicellular particulates. The tumor particulates each measure from about 0.25 to about 1.5 mm³, for example, about 1 mm³. After the tissue sample has been minced, the particles are plated in culture flasks. The number of explants plated per flask may vary, for example, between one and 25, such as from 5 to 20 explants per flask. For example, about 9 explants may be plated per T-25 flask, and 20 particulates may be plated per T-75 flask. For purposes of illustration, the explants may be evenly distributed across the bottom surface of the flask, followed by initial inversion for about 10-15 minutes. The flask may then be placed in a non-inverted position in a 37° C. CO₂ incubator for about 5-10 minutes. Flasks are checked regularly for growth and contamination. Over a period of days to a few weeks a cell monolayer will form.

Further, it is believed that malignant cells grow out from the multicellular explant prior to stromal cells. Thus, by initially maintaining the tissue cells within the explants and removing the explants at a predetermined time (e.g., at about 10 to about 50 percent confluency, or at about 15 to about 25 percent confluency), growth of the tumor cells (as opposed to stromal cells) into a monolayer is facilitated. In certain embodiments, the tumor explants may be agitated to substantially loosen or release tumor cells from the tumor explants, and the released cells cultured to produce a cell culture monolayer. The use of this procedure to form a cell culture monolayer helps maximize the growth of representative malignant cells from the tissue sample. Monolayer growth rate and/or cellular morphology (e.g., epithelial character) may be monitored using, for example, a phase-contrast inverted microscope. In some embodiments, the cells may be sub-cultured. Generally, the cells of the monolayer should be actively growing at the time the cells are suspended for RNA extraction.

The process for enriching or expanding malignant cells in culture is described in U.S. Pat. Nos. 5,728,541, 6,900,027, 6,887,680, 6,933,129, 6,416,967, 7,112,415, 7,314,731, 7,642,048, 7,501,260, and 7,642,048 (all of which are hereby incorporated by reference in their entireties). The process may further employ the variations described in US Published Patent Application No. 2007/0059821, which is hereby incorporated by reference in its entirety.

The breast tumors may be classified into estrogen receptor positive (ER+) and negative (ER−) subtypes by any suitable method, including immunohistochemistry or other immunoassay with antibody against ER. Alternatively, ER status may be determined by ER+ or ER− gene expression signatures, as described, for example, in Gruvberger, S. et al., (2001) Estrogen receptor status in breast cancer is associated with remarkably distinct gene expression patterns. Cancer Res., 61, 5979-5984; West, M. et al. (2001) Predicting the clinical status of human breast cancer by using gene expression profiles. Proc. Natl Acad. Sci. USA, 98, 11462-11467; and Kun Yu et al., Classifying the estrogen receptor status of breast cancers by expression profiles reveals a poor prognosis subpopulation exhibiting high expression of the ERBB2 receptor, Human Molecular Genetics 12(24):3245-3258 (2003).

In preparing the gene expression profile, RNA is extracted from the tumor tissue or cultured cells by any known method. For example, RNA may be purified from cells using a variety of standard procedures as described, for example, in RNA Methodologies, A laboratory guide for isolation and characterization, 2nd edition, 1998, Robert E. Farrell, Jr., Ed., Academic Press. In addition, there are various products commercially available for RNA isolation which may be used. Total RNA or polyA+ RNA may be used for preparing gene expression profiles in accordance with the invention.

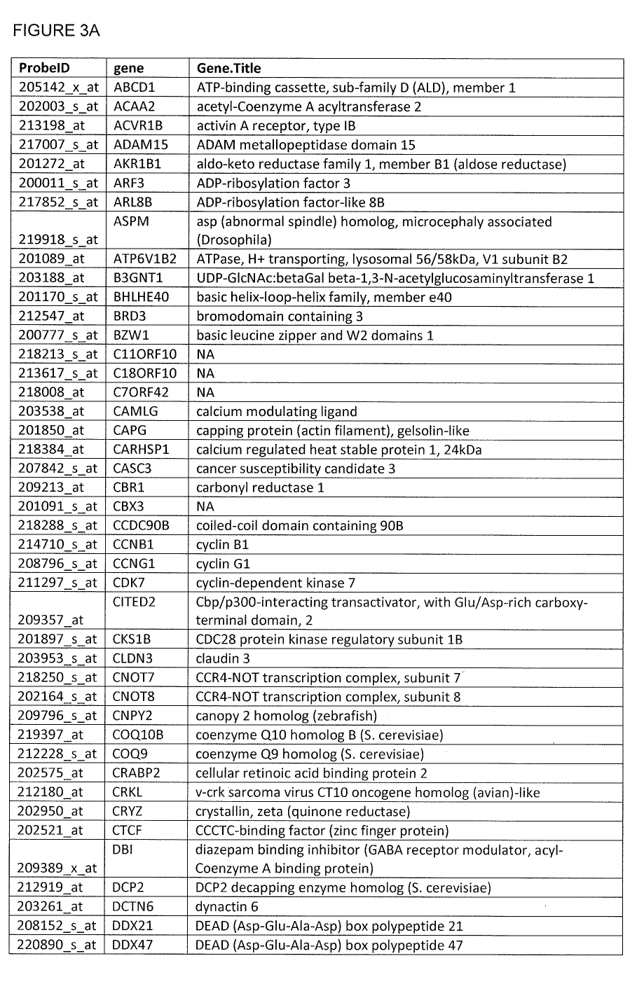

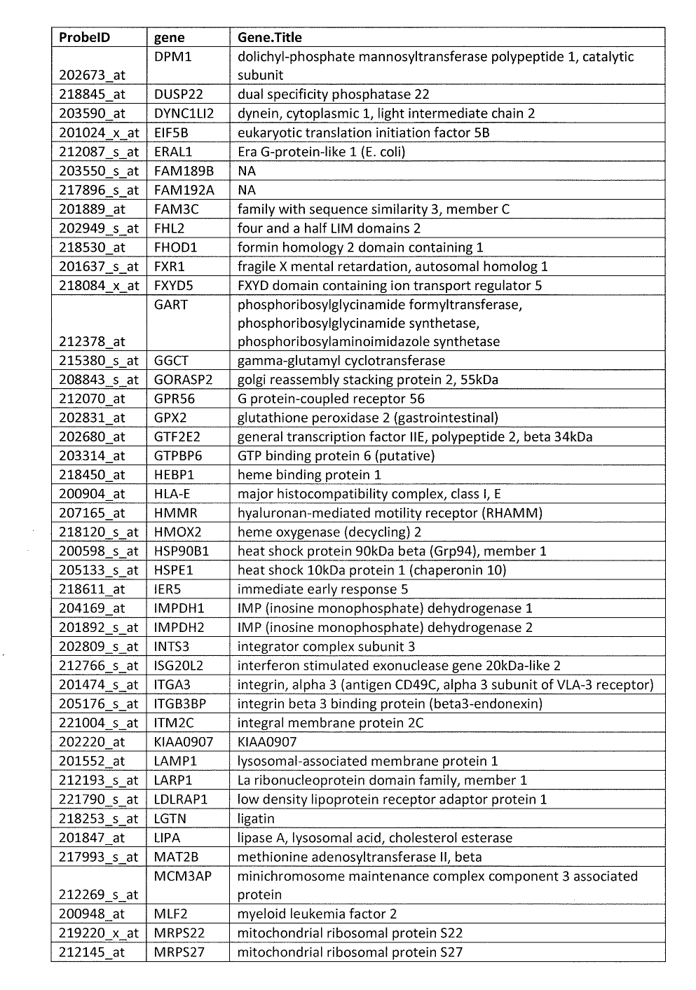

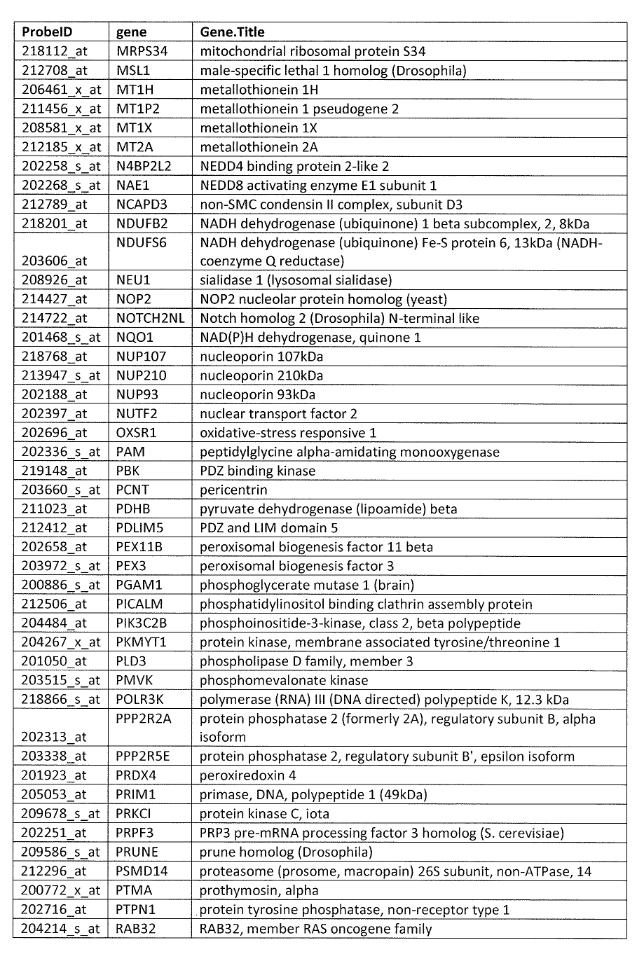

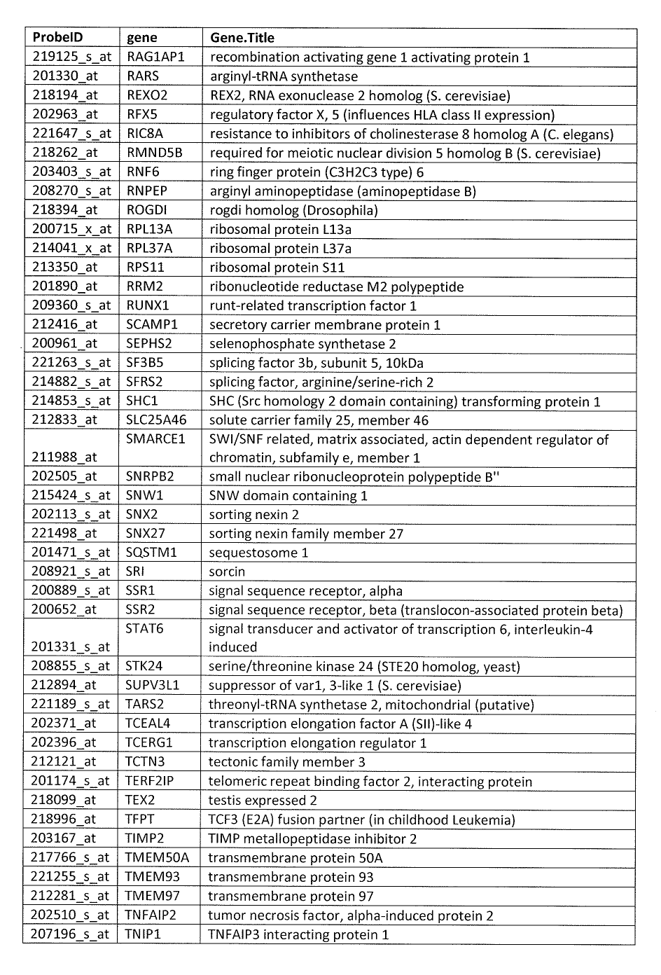

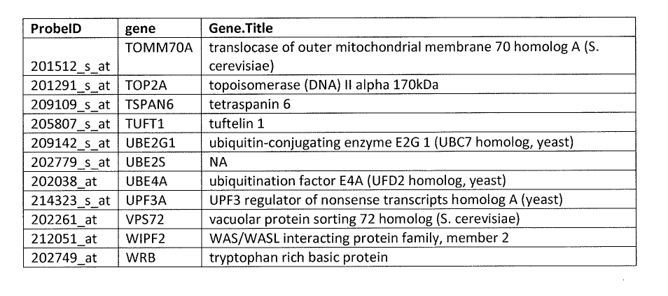

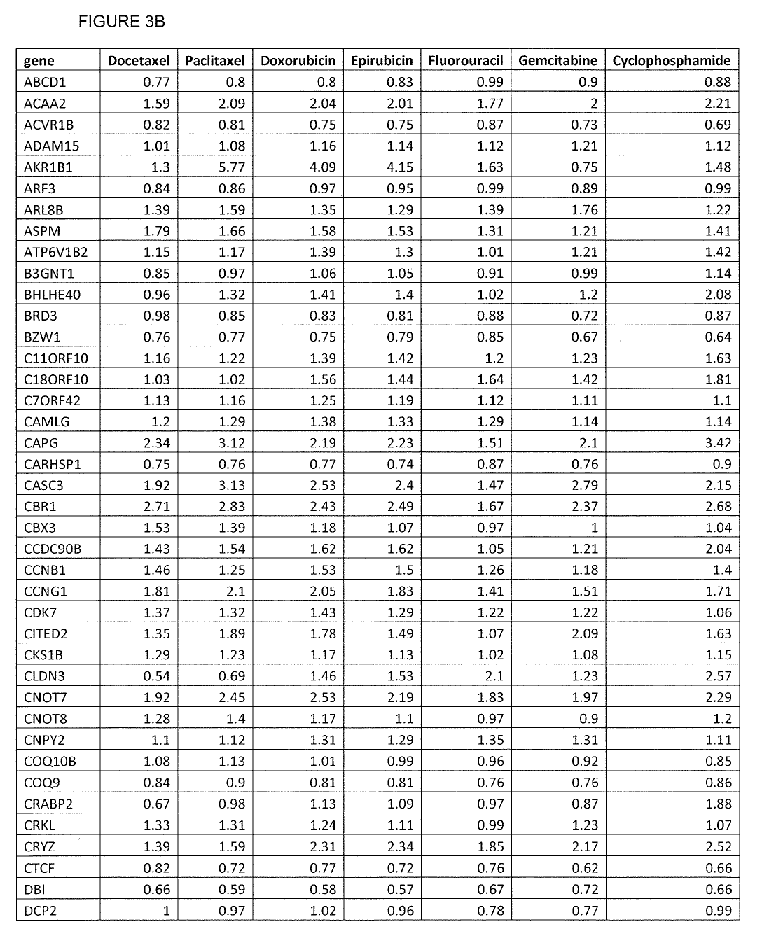

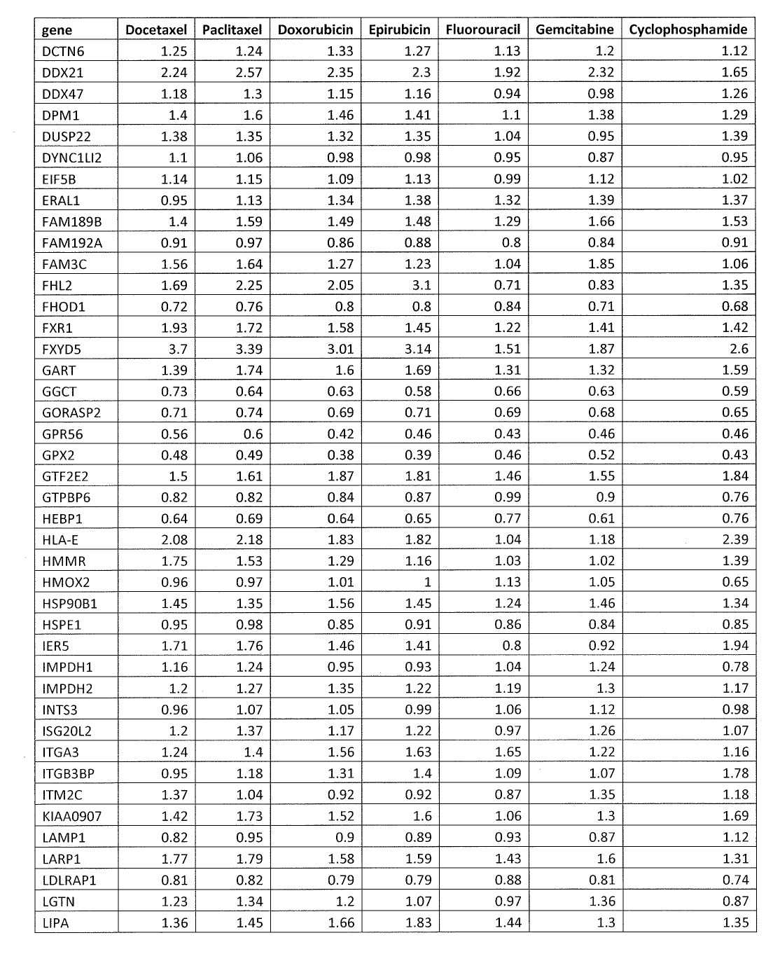

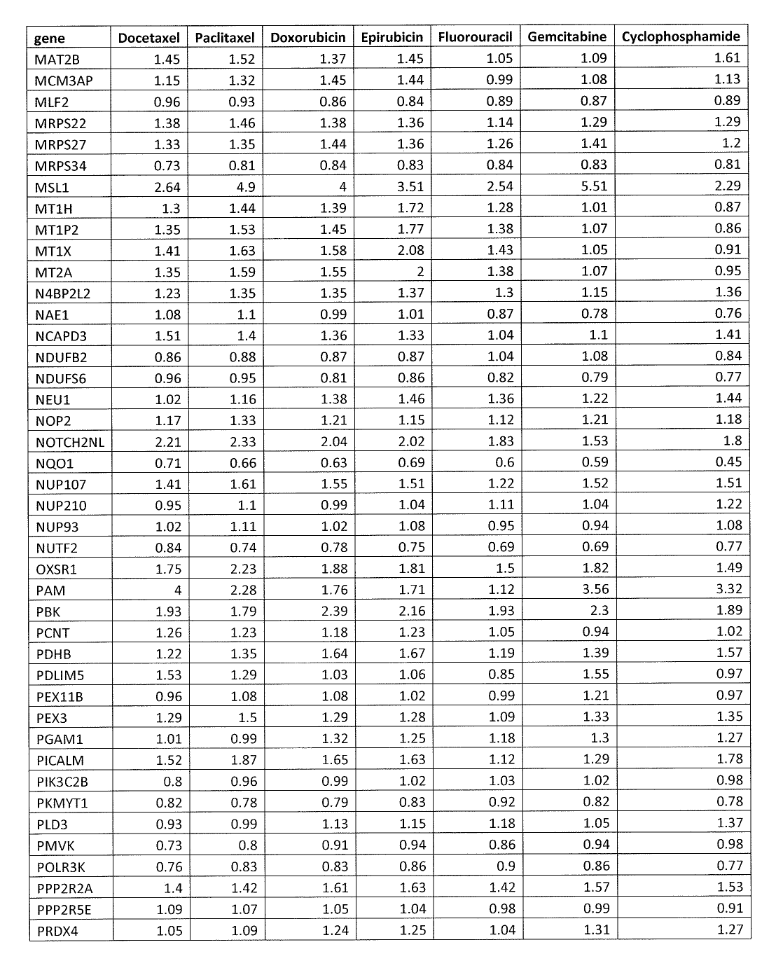

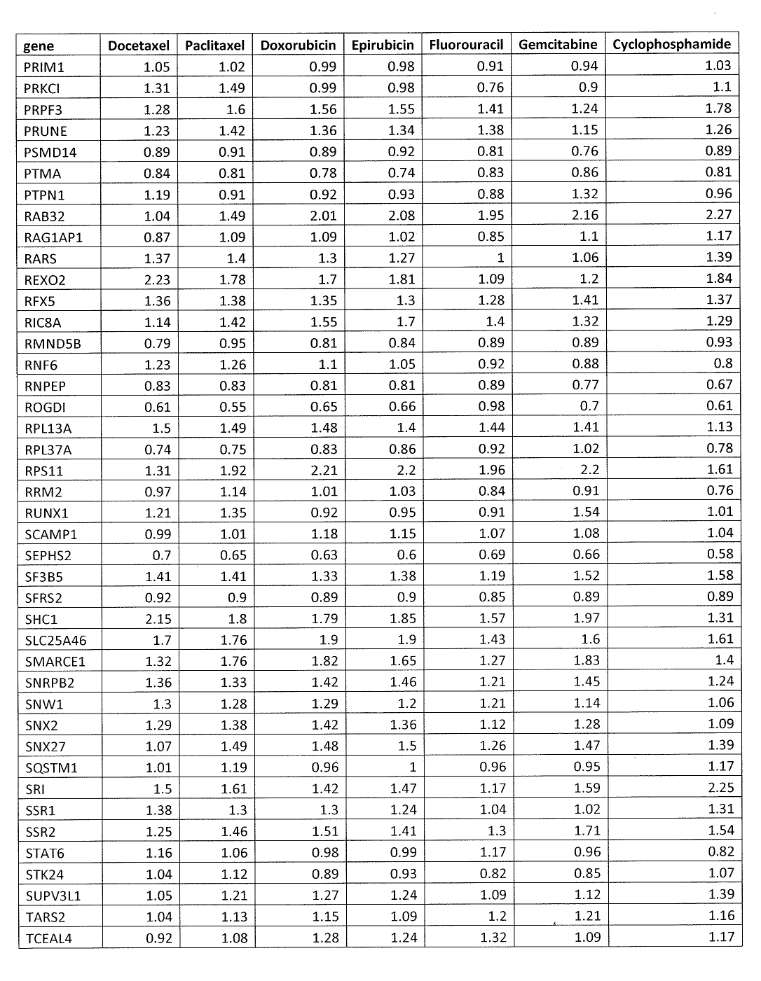

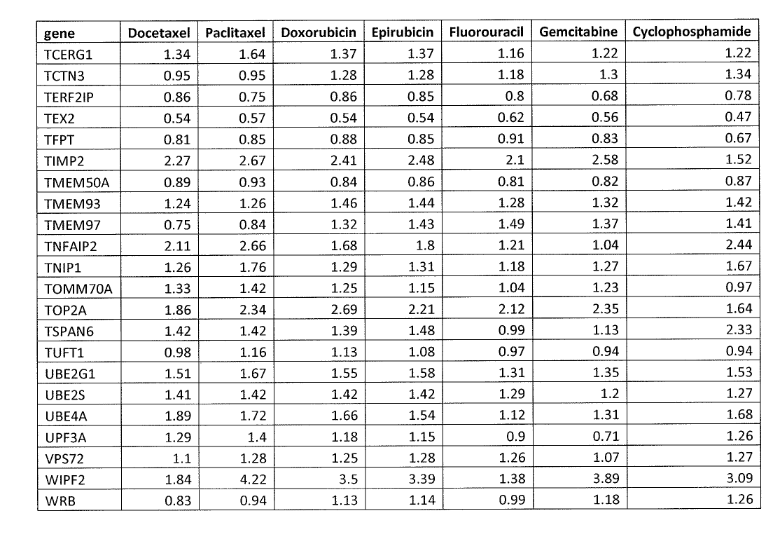

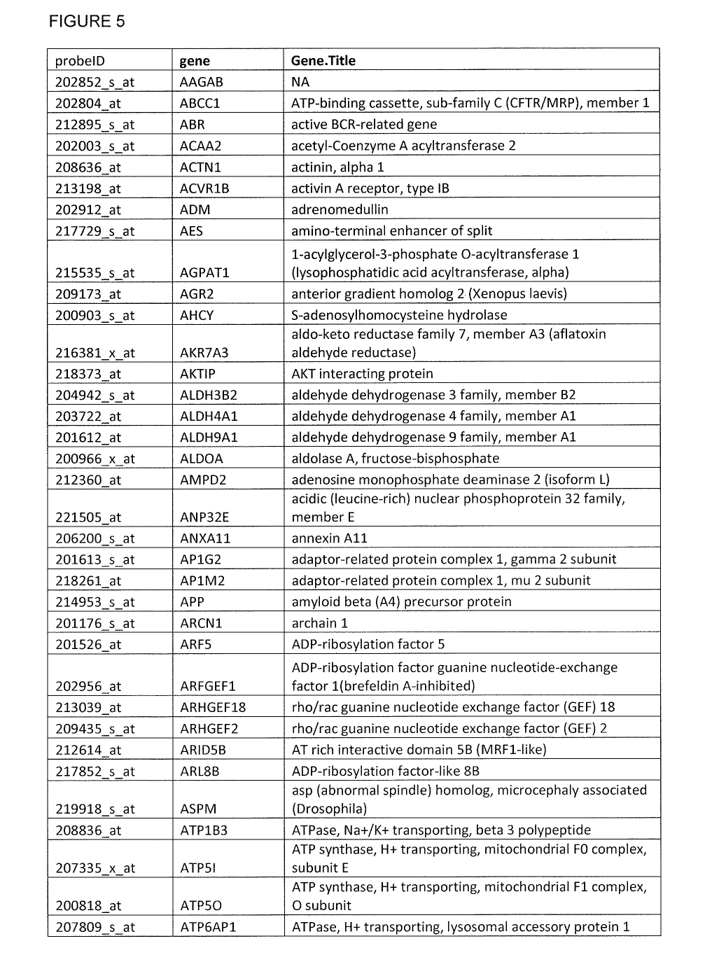

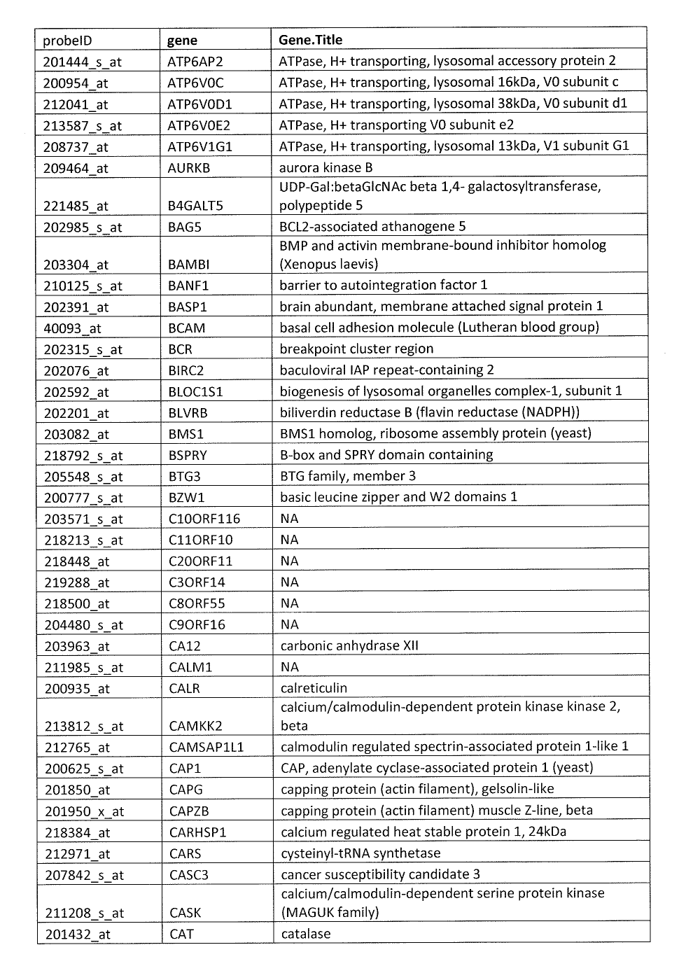

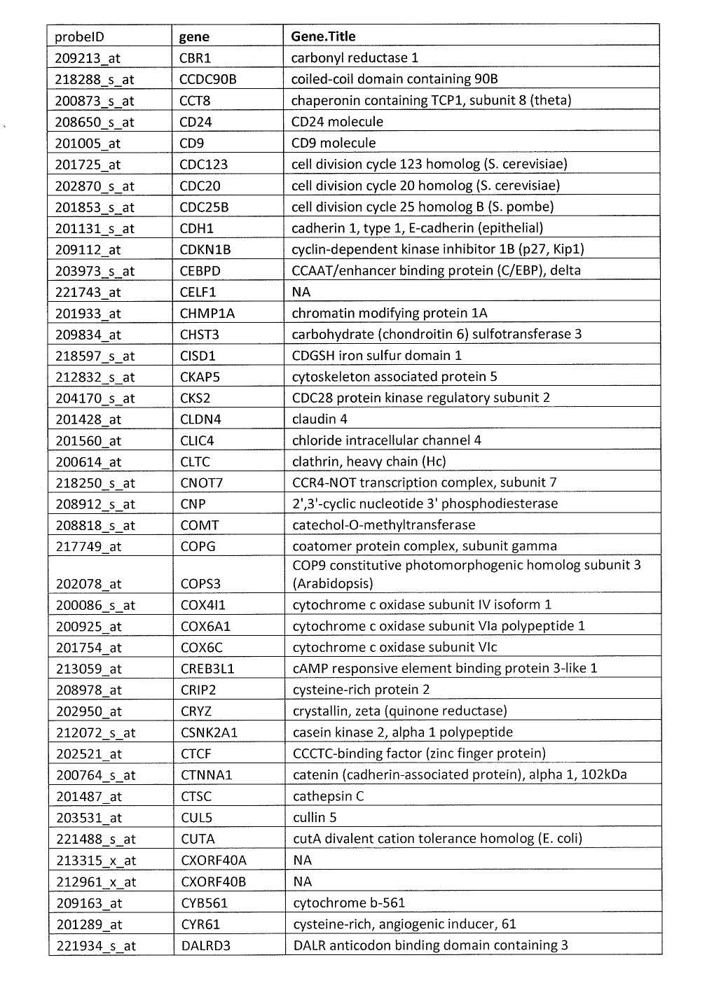

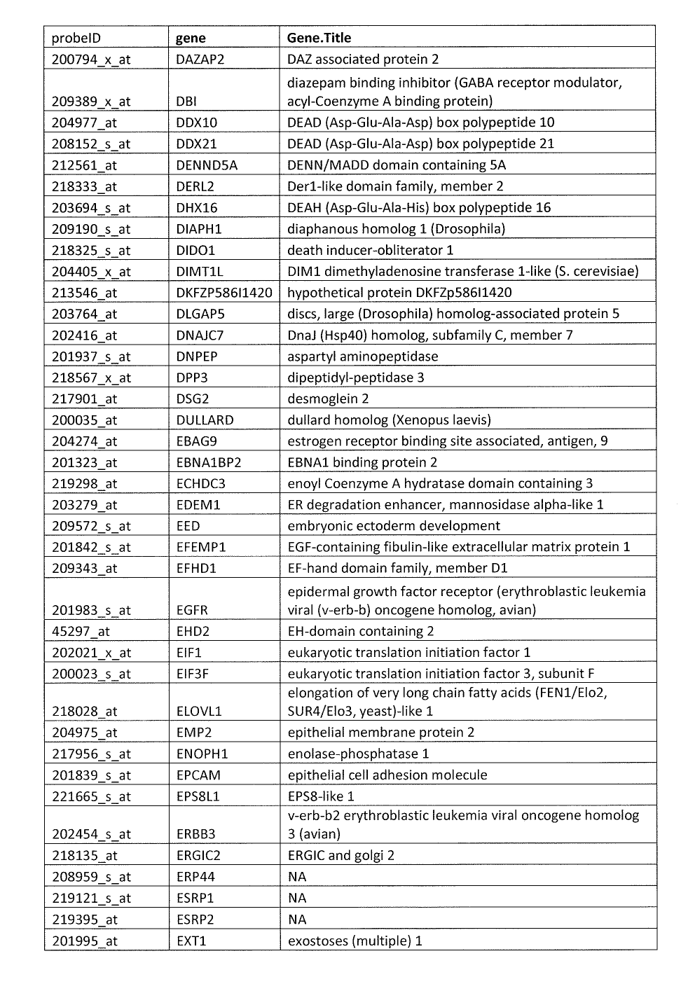

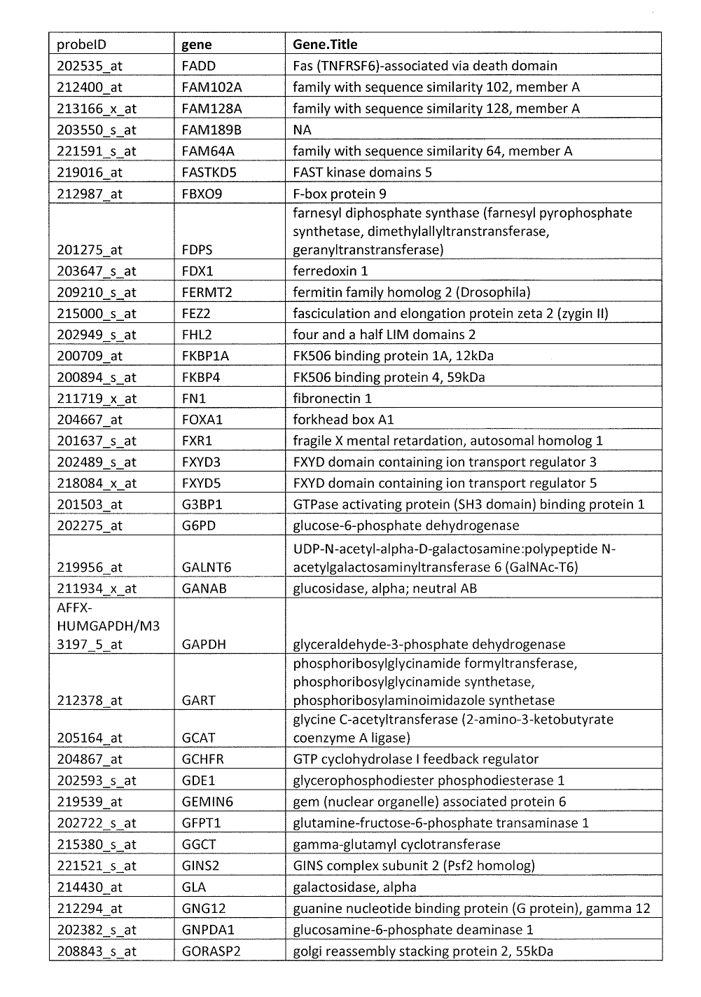

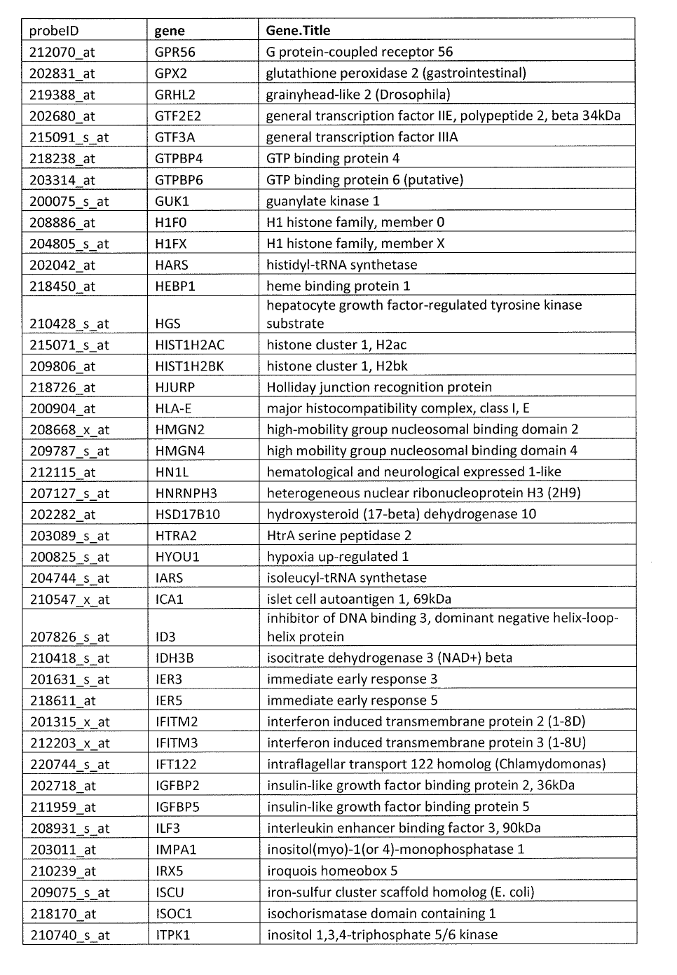

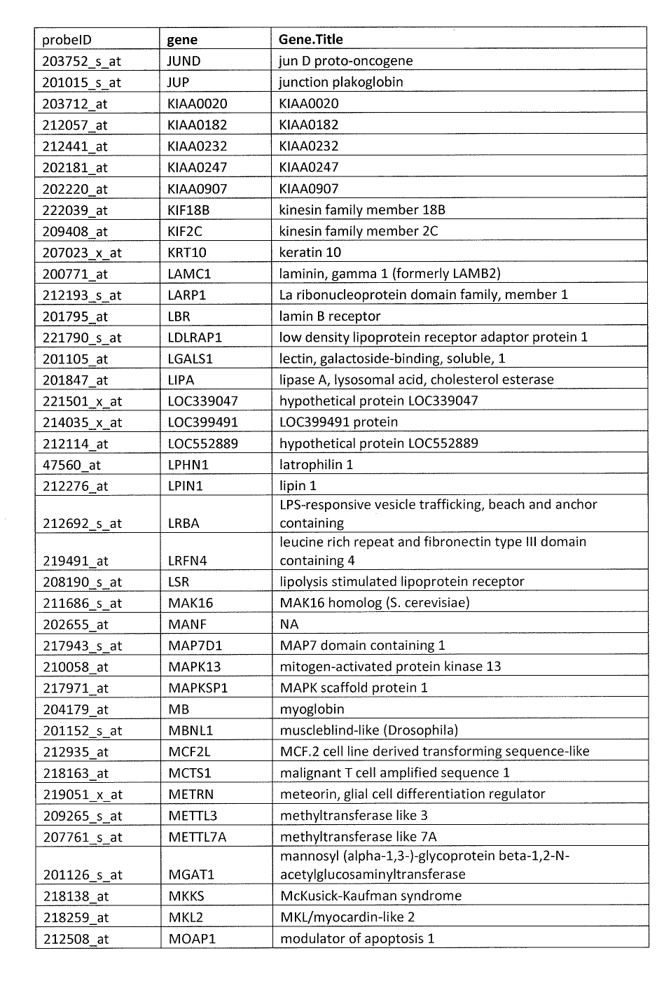

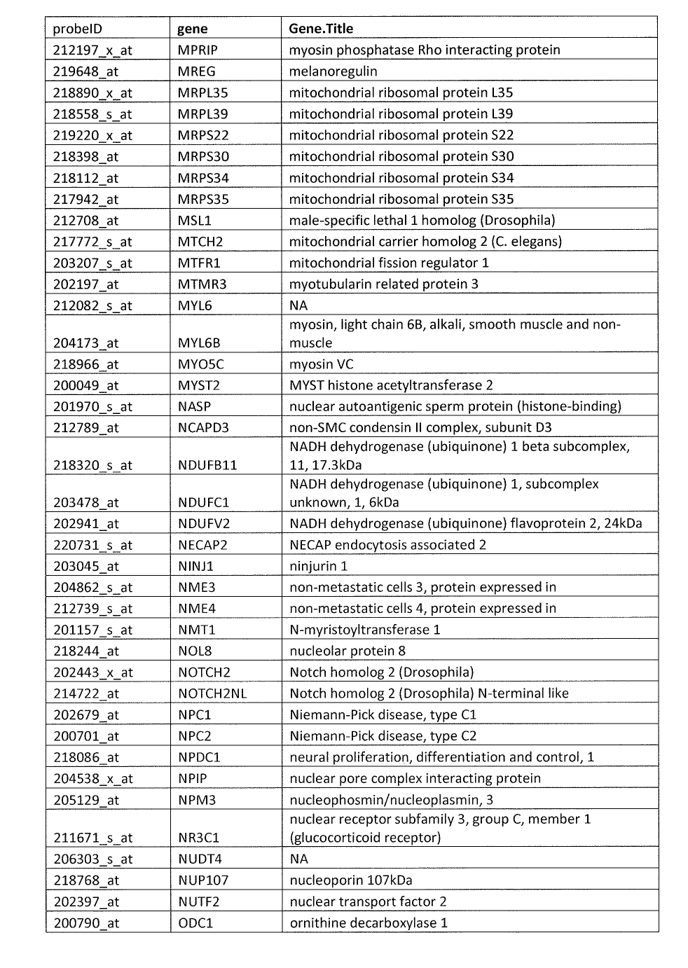

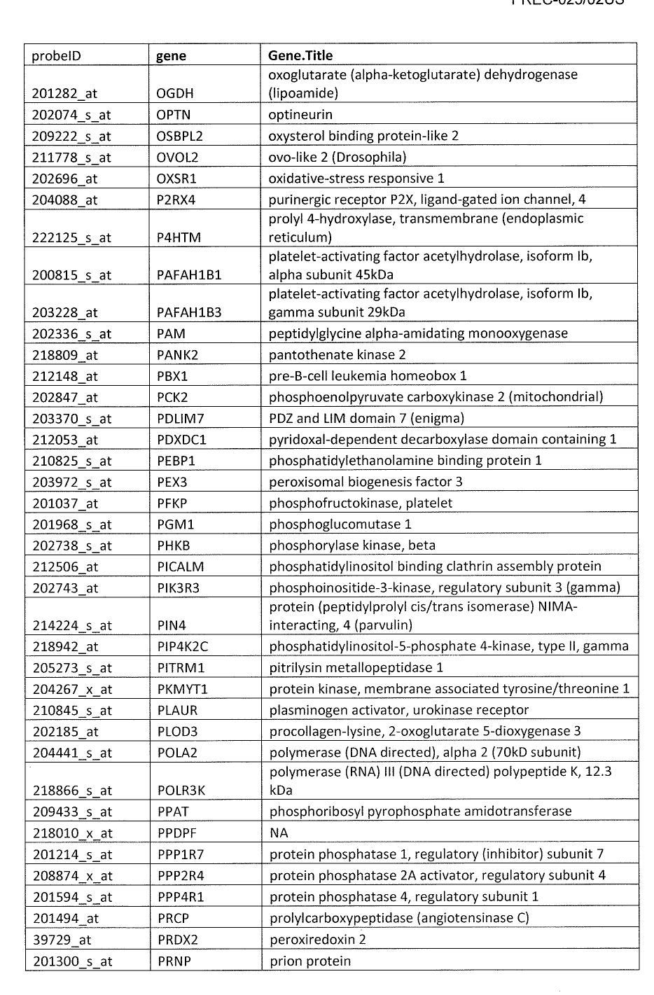

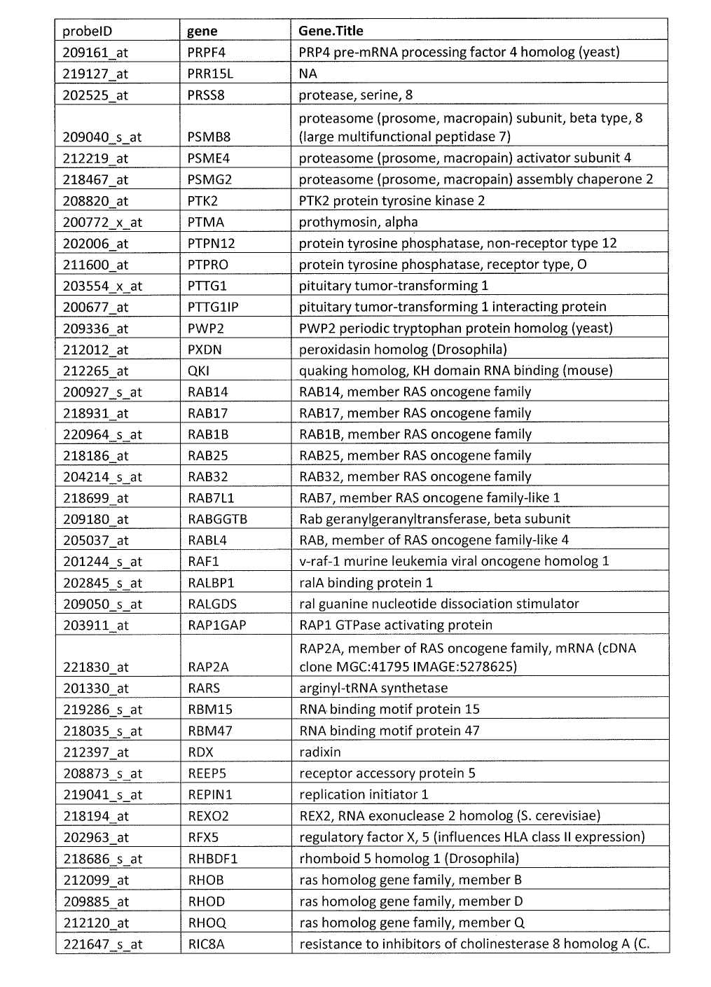

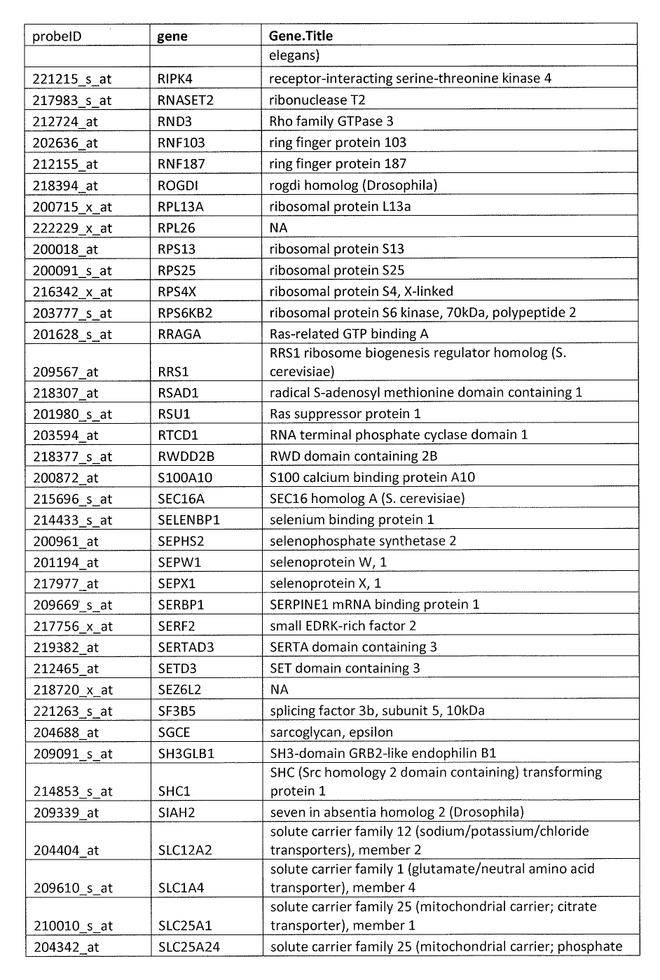

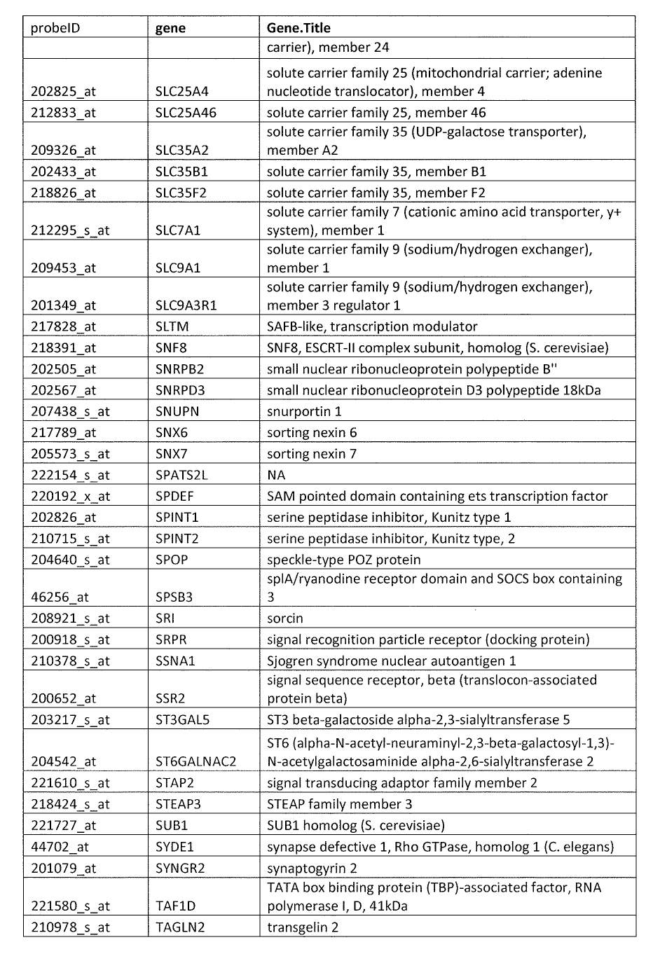

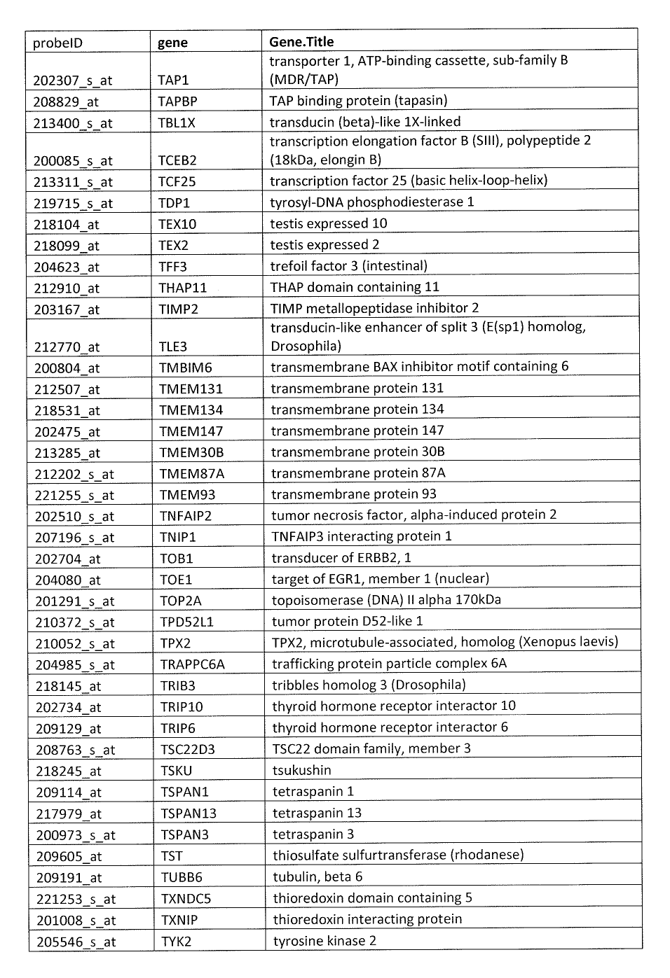

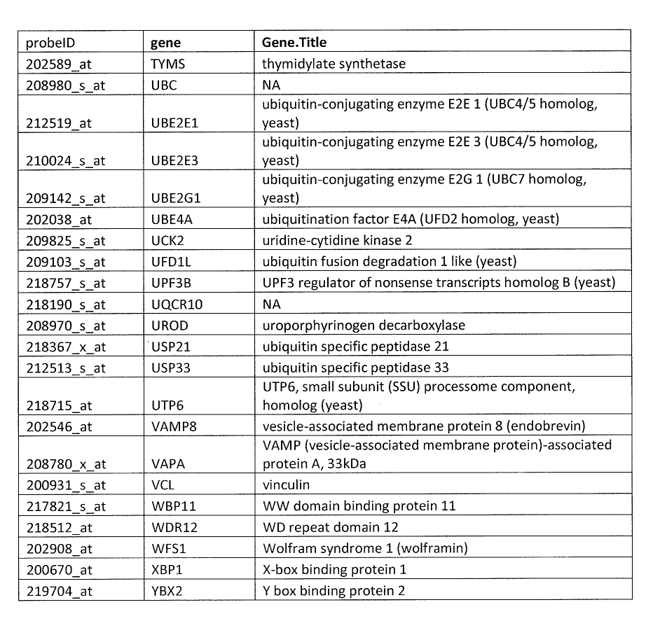

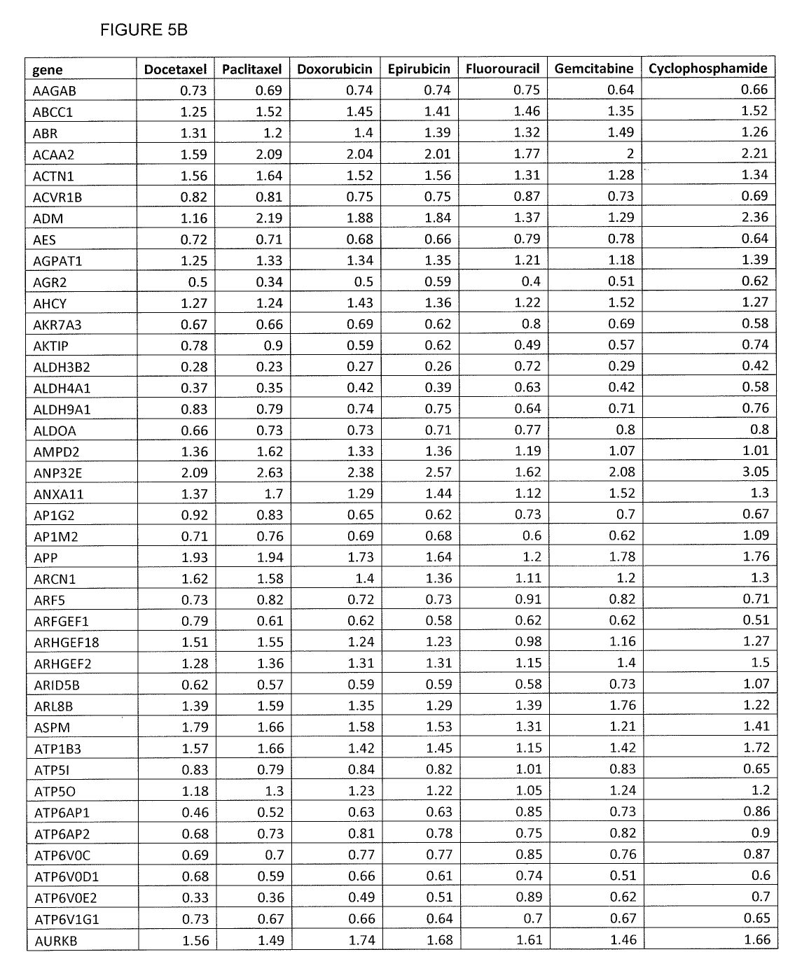

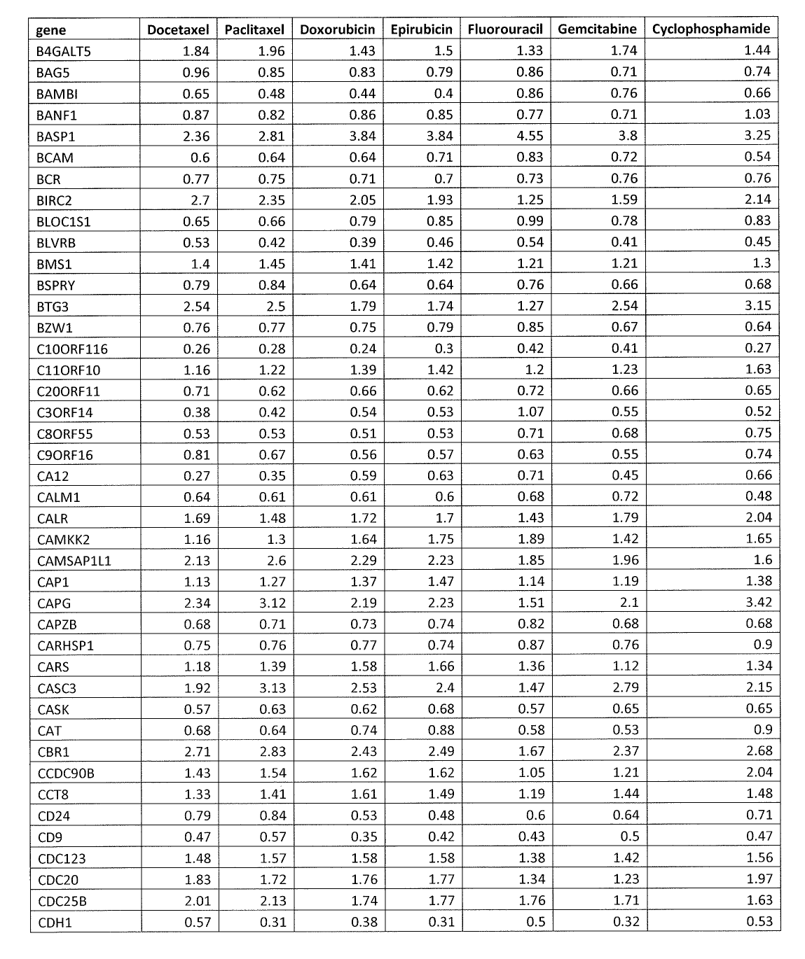

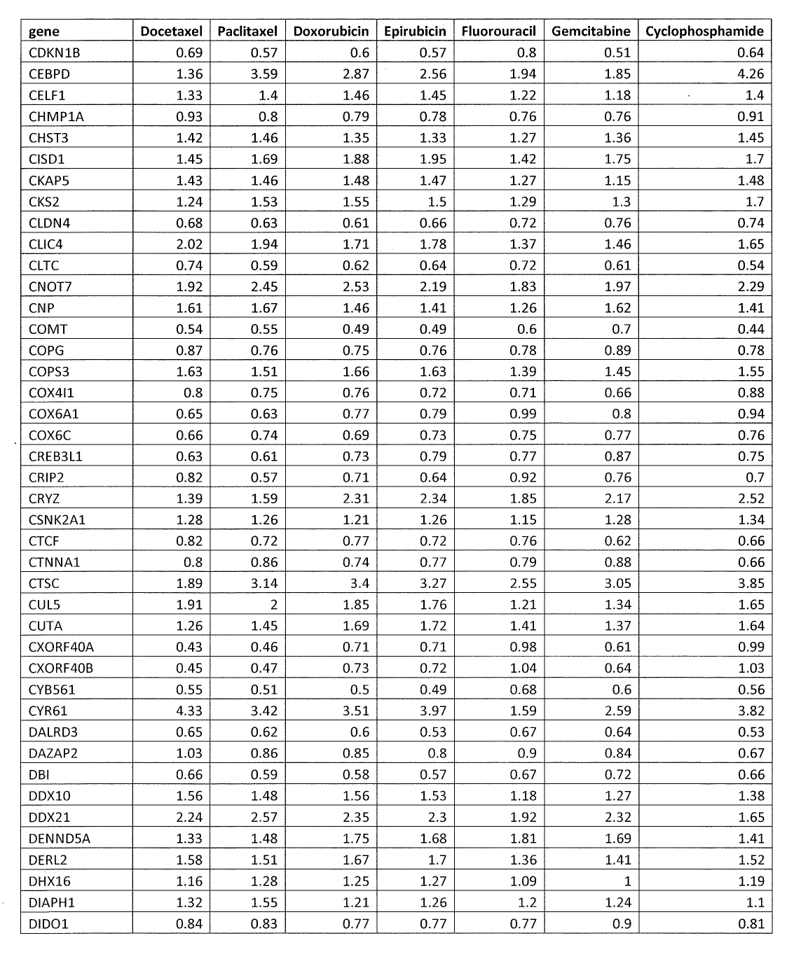

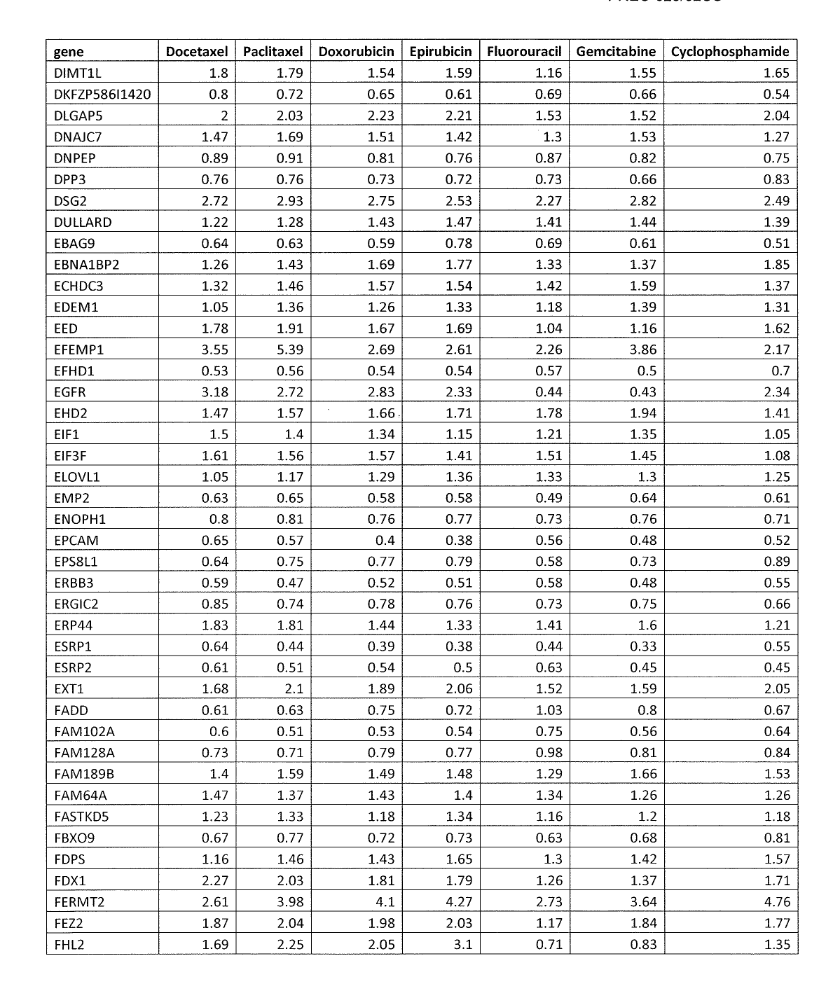

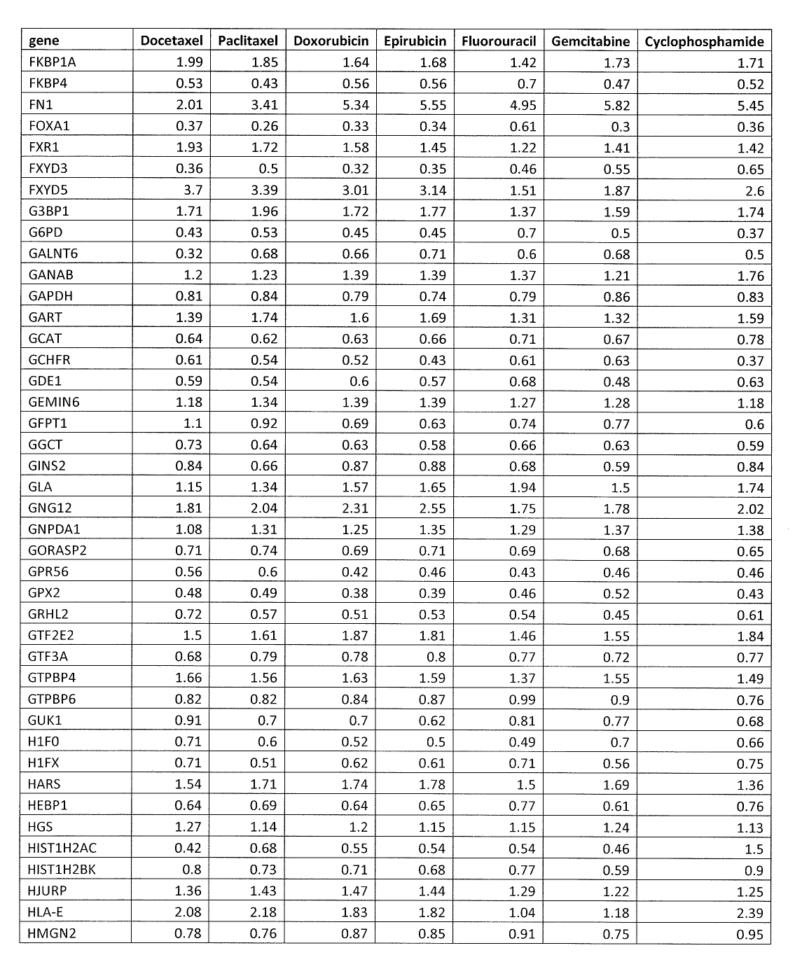

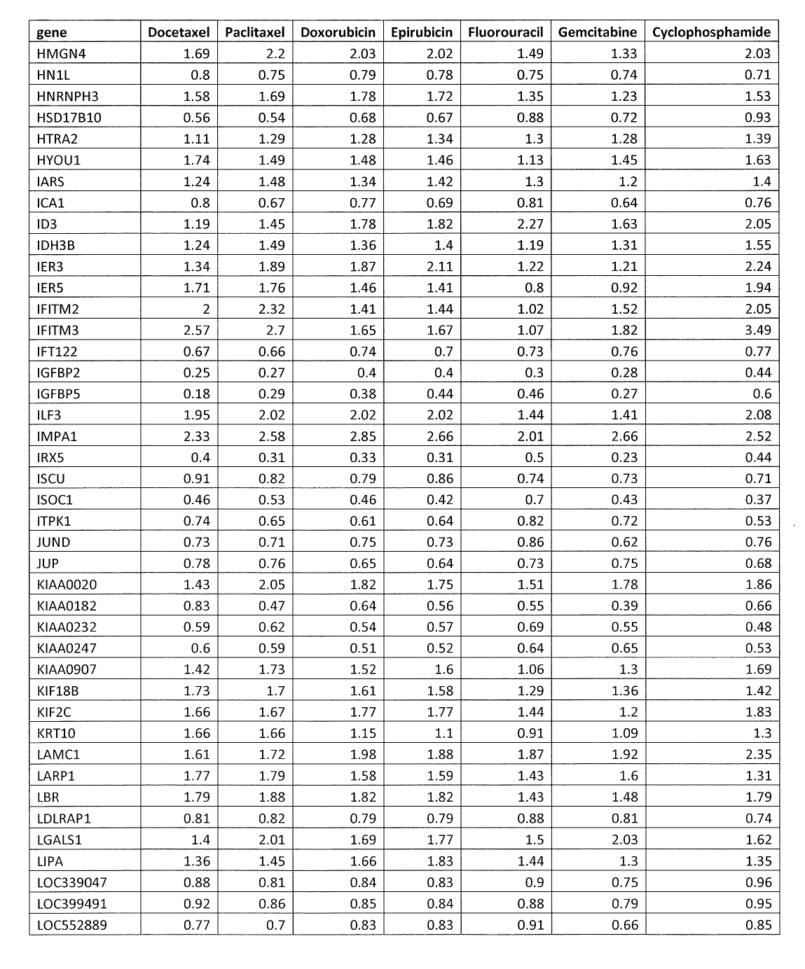

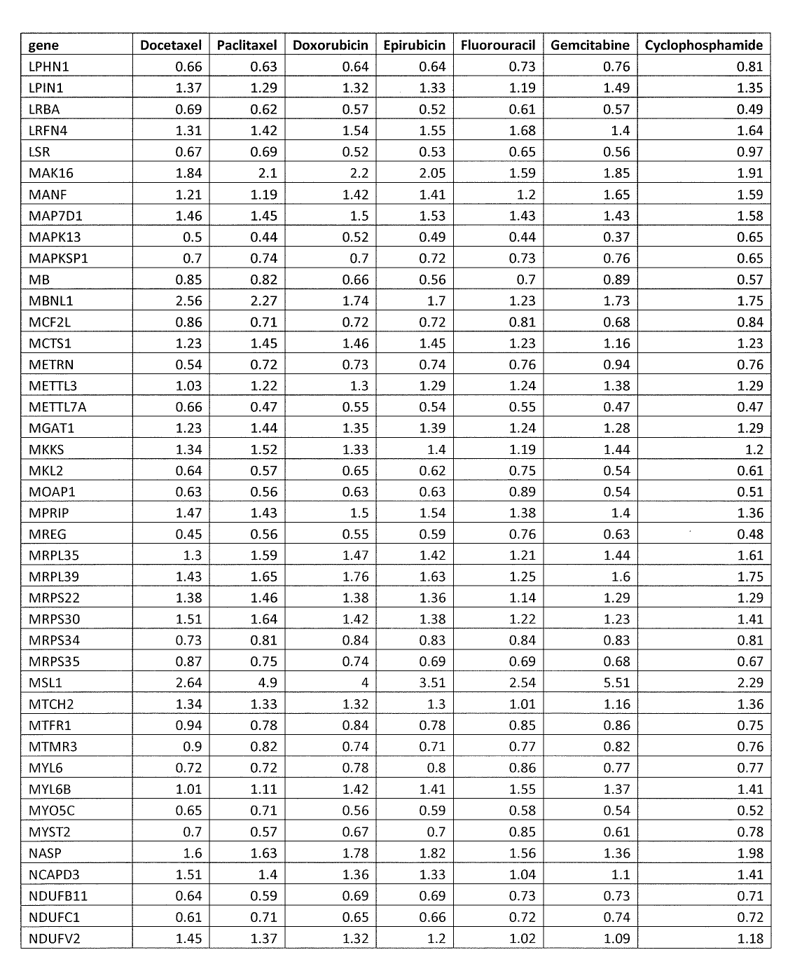

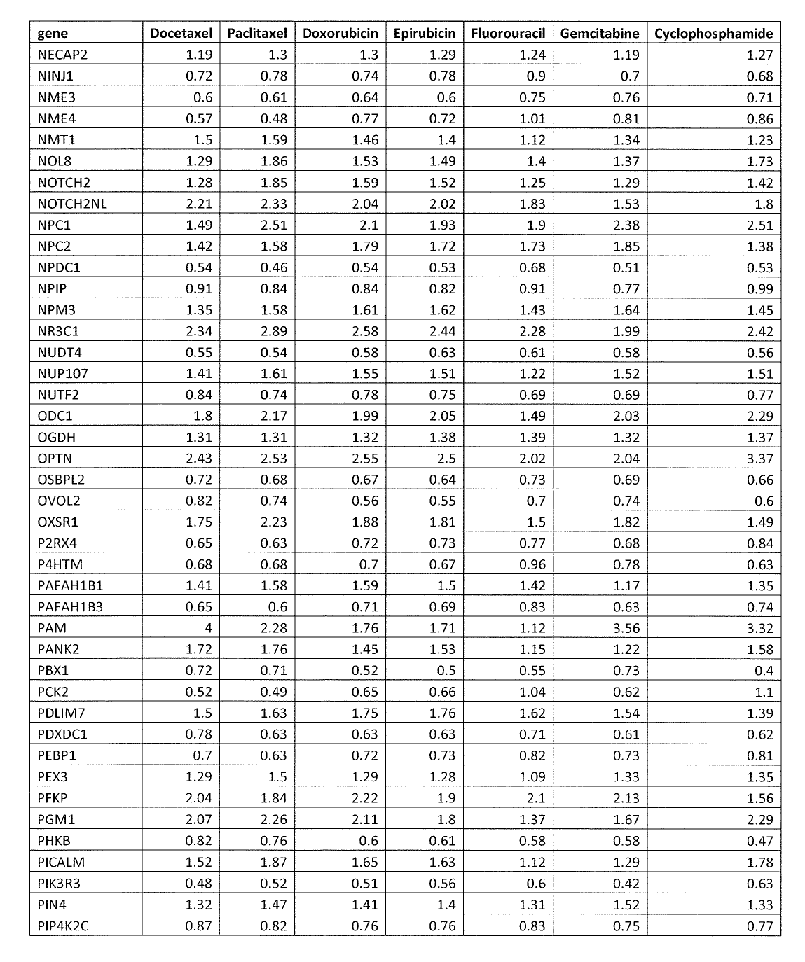

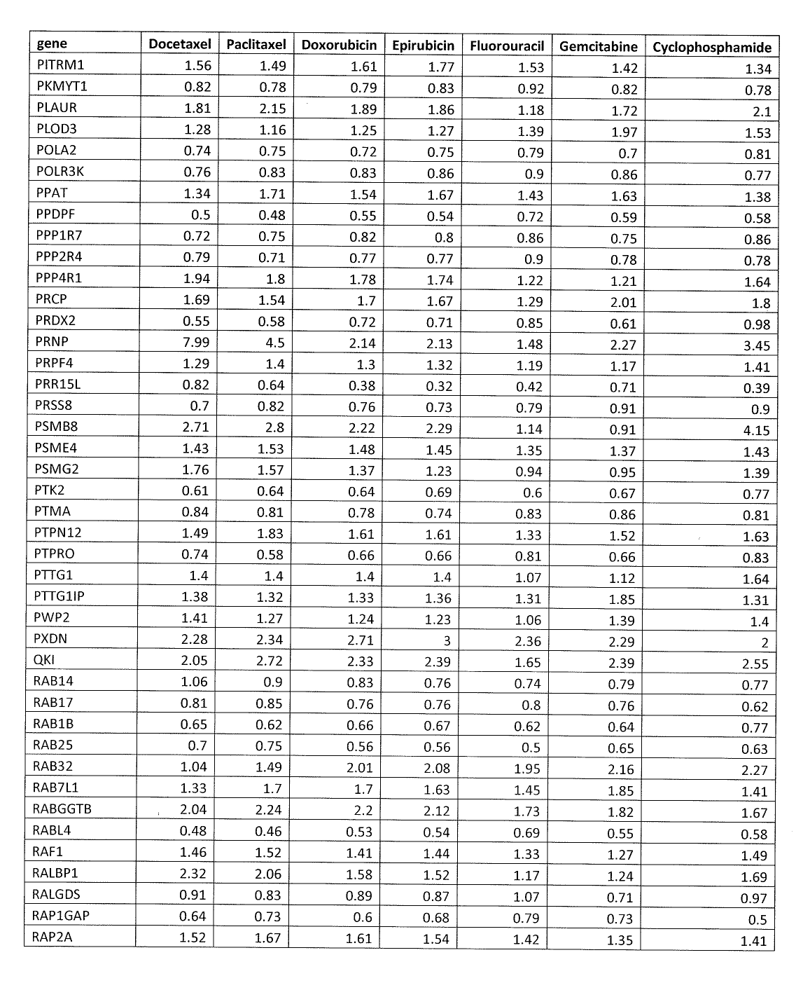

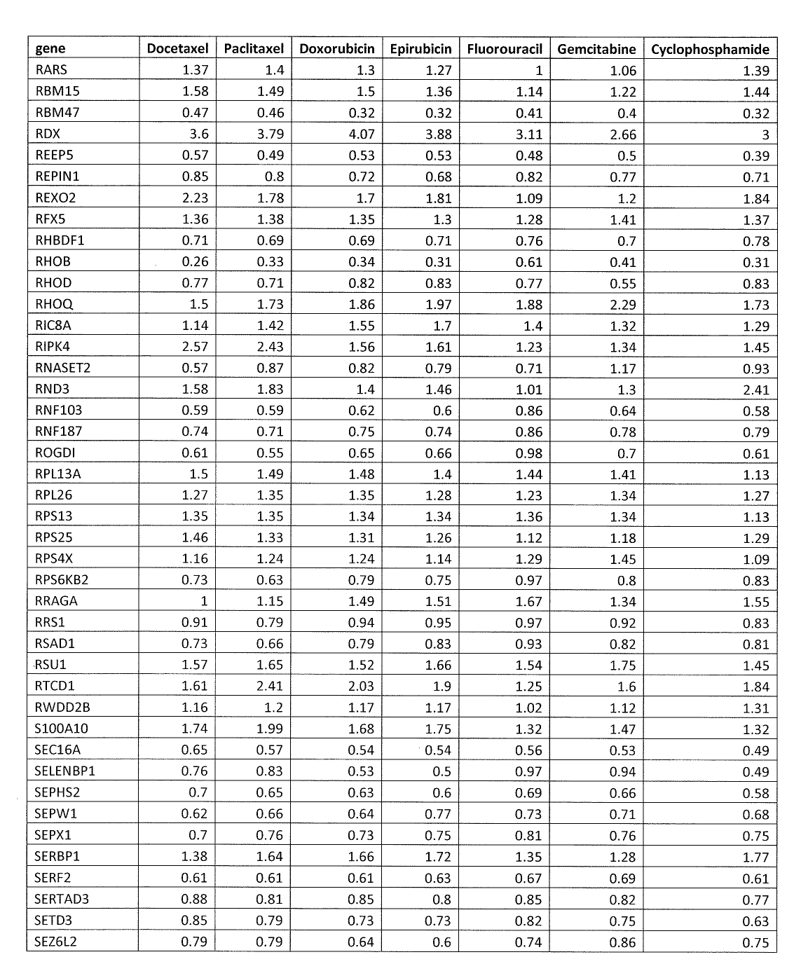

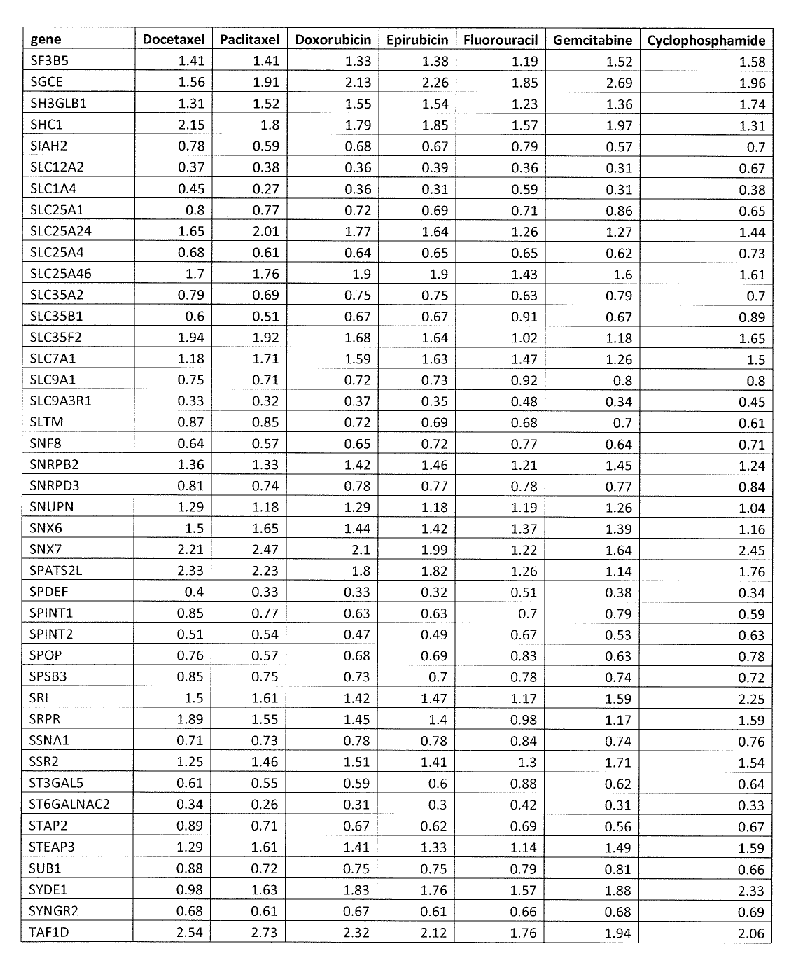

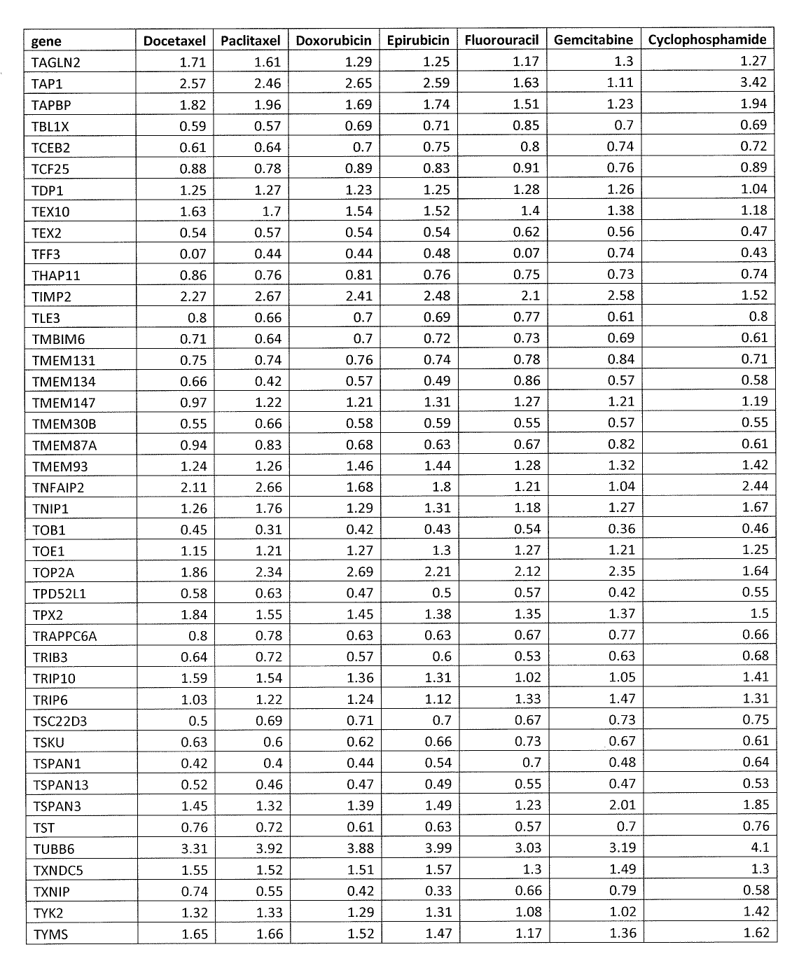

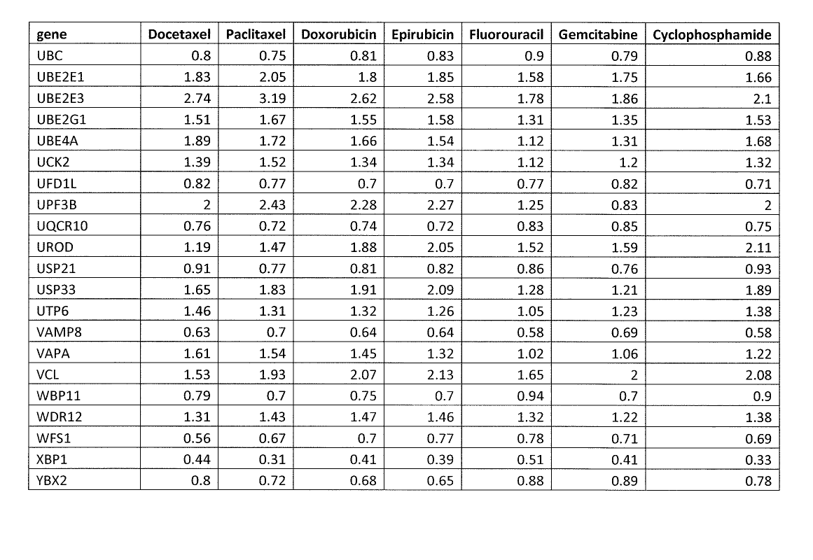

The gene expression profile is then generated for the samples using any of various techniques known in the art. Such methods generally include, without limitation, hybridization-based assays, such as microarray analysis and similar formats (e.g., Whole Genome DASL™ Assay, Illumina, Inc.), polymerase-based assays, such as RT-PCR (e.g., Taqman™), flap-endonuclease-based assays (e.g., Invader™), as well as direct mRNA capture with branched DNA (QuantiGene™) or Hybrid Capture™ (Digene). In certain embodiments, the gene expression profile is determined using a microarray format, such as the Affymetrix HGU133A, or relevant probes therefrom. The polynucleotide sequences of the genes listed in FIGS. 3-5 are publicly available, and are hereby incorporated by reference. Further, Affymetrix probe sequences for such genes, as employed with the HGU133A array, are also hereby incorporated by reference.

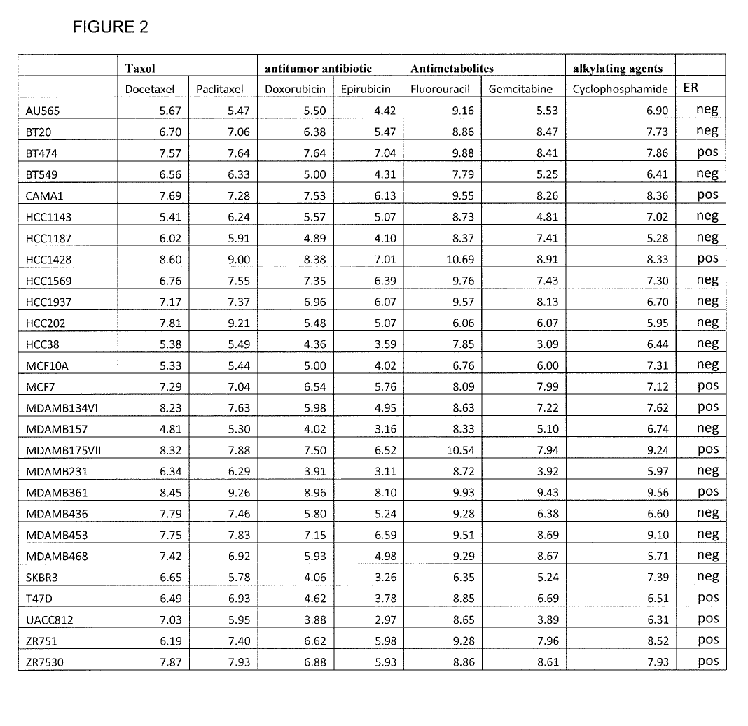

The gene expression profile contains gene expression levels for a plurality of genes whose expression levels are predictive or indicative of the tumor's resistance to one or a combination of chemotherapeutic agents. The gene expression signatures can be defined by the level of gene expression exhibited by drug-sensitive breast cancer cell lines (immortal cell lines), versus the level of gene expression exhibited by drug-resistant (multi drug-resistant) breast cancer cell lines (immortal cell lines). Drug-sensitive and drug-resistant cell lines are defined by their drug response in an in vitro chemosensitivity assay (described herein). In certain embodiments, the gene expression signature is defined by the gene expression levels of the breast cancer cell lines of FIG. 2, as grouped according to their chemosenstivity profile and/or ER status.

As used herein, the term “gene,” refers to a DNA sequence expressed in a sample as an RNA transcript, and may be a full-length gene (protein encoding or non-encoding) or an expressed portion thereof such as expressed sequence tag or “EST.” Thus, the genes listed in FIGS. 3-5 are each independently a full-length gene sequence, whose expression product is present in samples, or is a portion of an expressed sequence detectable in samples, such as an EST sequence.

The genes listed in FIGS. 3-5 may be differentially expressed in drug-sensitive cells versus drug-resistant cells (e.g., multidrug resistant samples). As used herein, “differentially expressed” means that the level or abundance of an RNA transcript (or abundance of an RNA population sharing a common target (or probe-hybridizing) sequence, such as a group of splice variant RNAs) is significantly higher or lower in a sample (e.g., a drug-resistant sample) as compared to a reference level (e.g., a drug sensitive sample). For example, the level of the RNA or RNA population may be higher or lower than a reference level. The reference level may be the level of the same RNA or RNA population in a control sample or control population (e.g., a Mean or Median level for a drug-sensitive cell), or may represent a cut-off or threshold level for a sensitive or resistant designation.

The gene expression profile generally contains the expression levels for at least about 3, 5, 7, 10, 25, 50, 100 or more (e.g., all or substantially all) genes listed in one or more of FIGS. 3-5. As discussed, the expression levels for these genes represent the gene expression state of the patient's malignant cells or tumor, and together this profile is evaluated for the presence of one or more gene signatures indicative of the tumor's sensitivity and/or resistance to chemotherapeutic agents. In some embodiments, the profile is prepared with the use of a custom array or bead set (or other gene expression detection format), so as to quantify the level of 500 genes of less, 250 genes or less, 150 genes or less, or 100 genes or less, including genes listed in FIGS. 3-5.

Alternatively or in addition, the gene expression profile may contain the levels of expression for at least about 3 genes listed in FIG. 3. In some embodiments, the patient's gene expression profile contains the levels of expression for at least about 5, 7, 10, 12, 15, 20, 25, 50, or all genes listed in FIG. 3, such genes being differentially expressed in multidrug-resistant ER positive breast cancer cells versus drug sensitive ER positive breast cancer cells. In some embodiments, the gene expression profile may contain the levels of expression for all or substantially all genes listed in FIG. 3. In some embodiments, the profile is prepared with the use of a custom array or bead set (or other gene expression detection format), so as to quantify the level of 500 genes of less, 250 genes or less, 150 genes or less, or 100 genes or less, including genes listed in FIG. 3.

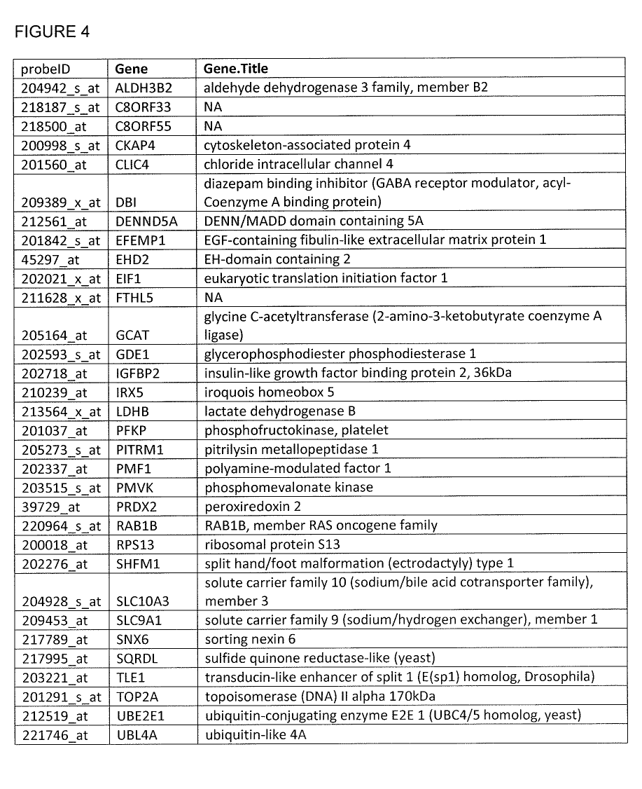

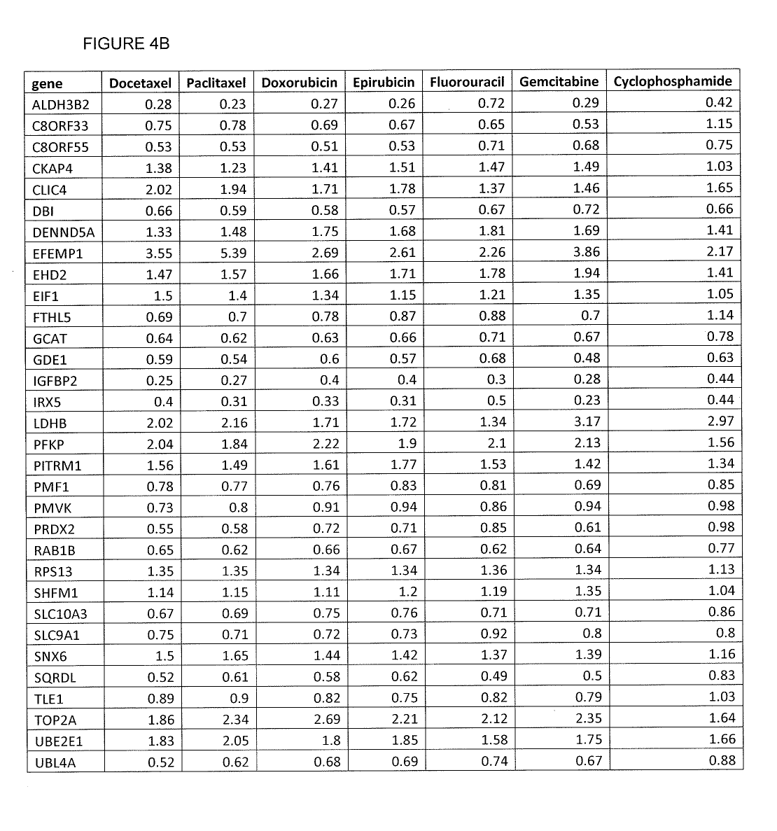

Alternatively or in addition, the gene expression profile may contain the levels of expression for at least about 3 genes listed in FIG. 4. In some embodiments, the patient's gene expression profile contains the levels of expression for at least about 5, 7, 10, 12, 15, 20, 25, or all genes listed in FIG. 4, such genes being differentially expressed in multidrug-resistant ER negative breast cancer cells versus drug sensitive ER negative breast cancer cells. In some embodiments, the gene expression profile may contain the levels of expression for all or substantially all genes listed in FIG. 4. In some embodiments, the profile is prepared with the use of a custom array or bead set (or other gene expression detection format), so as to quantify the level of 500 genes of less, 250 genes or less, 150 genes or less, or 100 genes or less, including genes listed in FIG. 4.

In certain embodiments, the gene expression profile contains a measure of expression level for the plurality of genes (e.g., 5, 7, 10, 12, 15, 50, etc.) that are each, independently, expressed in multidrug-sensitive versus drug-resistant samples by a fold change magnitude (up or down) of at least about 1.2 (up) or about 0.8 (down). Fold change magnitude is defined as mean sensitive score/mean resistant score. In some embodiments, the plurality of genes are differentially expressed in drug sensitive versus drug resistant cells by a fold change magnitude (up) of at least 1.5, or at least about 1.7, or at least about 2, or at least about 2.5, or by a fold magnitude (down) of less than about 0.7, about 0.5, or about 0.4. Alternatively, the expression levels (mean sensitive and mean resistant) may differ by at least about 2-, 3-, 4-, or 5-, 10-fold, or more.

The gene expression profile prepared according to this aspect of the invention is evaluated for the presence of one or more gene expression signatures indicative of drug responsiveness (e.g., a multidrug resistant signature). The gene expression signature(s) comprise or are derived from (mathematically) the gene expression levels indicative of a drug-sensitive and/or multidrug-resistant cells, so as to enable a classification of the tumor's profile as sensitive or resistant. Specifically, the gene expression signature comprises or is derived from indicative gene expression levels for a plurality of genes listed in one or more of FIGS. 3-5, such as at least 5, 7, 10, 12, 15, 20, 25, 40, 50, 75, 100, 200, 250, 300, 400, or 500 genes listed in one or more of FIGS. 3-5. The signature may comprise or be derived from the Mean or Median expression levels, or alternatively, may use other statistical criteria.

The gene expression signature(s) may be in a format consistent with any nucleic acid detection format, such as those described herein, and will generally be comparable to the format used for profiling patient samples. For example, the gene expression signature and patient profiles may both be prepared by nucleic acid hybridization method, and with the same hybridization platform and controls so as to facilitate comparisons. The gene expression signatures may further embody any number of statistical measures to distinguish drug-sensitive and/or drug-resistant levels, including Mean or median expression levels and/or cut-off or threshold values. Such signatures may be prepared from the data sets disclosed herein or independent gene expression data sets.

Once the gene expression profile for patient samples are prepared, the profile is evaluated for the presence of one or more of the gene signatures, by scoring or classifying the patient profile against each gene signature.

Various classification schemes are known for classifying samples between two or more classes or groups, and these include, without limitation: Principal Components Analysis, Naïve Bayes, Support Vector Machines, Nearest Neighbors, Decision Trees, Logistic, Artificial Neural Networks, and Rule-based schemes. In addition, the predictions from multiple models can be combined to generate an overall prediction. For example, a “majority rules” prediction may be generated from the outputs of a Naïve Bayes model, a Support Vector Machine model, and a Nearest Neighbor model.

Thus, a classification algorithm or “class predictor” may be constructed to classify samples. The process for preparing a suitable class predictor is reviewed in R. Simon, Diagnostic and prognostic prediction using gene expression profiles in high-dimensional microarray data, British Journal of Cancer (2003) 89, 1599-1604, which review is hereby incorporated by reference in its entirety.

Generally, the gene expression profiles for patient specimens are scored or classified as drug-sensitive signatures or drug-resistant signatures, including with stratified or continuous intermediate classifications or scores reflective of drug resistance or sensitivity. As discussed, such signatures may be assembled from publicly available gene expression data, or prepared from independent data sets. The signatures may be stored in a database and correlated to patient tumor gene expression profiles in response to user inputs.

After comparing the patient's gene expression profile to the drug-sensitive and/or drug-resistant signature, the sample is classified as, or for example, given a probability of being, a drug-sensitive profile or a drug-resistant profile (e.g., a multidrug resistant profile). The classification may be determined computationally based upon known methods as described above. The result of the computation may be displayed on a computer screen or presented in a tangible form, for example, as a probability (e.g., from 0 to 100%) of the patient responding to a given treatment. The report will aid a physician in selecting a course of treatment for the cancer patient. For example, in certain embodiments of the invention, the patient's gene expression profile will be determined to be a drug-sensitive profile on the basis of a probability, and the patient will be subsequently treated with that drug or combination. In other embodiments, the patient's profile will be determined to be a drug-resistant profile, such as a multidrug resistant profile, thereby allowing the physician to exclude one or more candidate treatments for the patient, thereby sparing the patient the unnecessary toxicity.

In various embodiments, the method according to this aspect of the invention distinguishes a drug-sensitive tumor from a multidrug-resistant tumor with at least about 60%, 75%, 80%, 85%, 90%, 95% or greater accuracy. In this respect, the method according to this aspect may lend additional or alternative predictive value over standard methods, such as for example, gene expression tests known in the art, or chemoresponse testing.

The methods of the invention aid the prediction of an outcome of treatment, e.g., based on a probability. That is, the gene expression signatures are each predictive of an outcome upon treatment with a candidate agent or combination. The outcome may be quantified in a number of ways. For example, the outcome may be an objective response, a clinical response, or a pathological response to a candidate treatment. The outcome may be determined based upon the techniques for evaluating response to treatment of solid tumors as described in Therasse et al., New Guidelines to Evaluate the Response to Treatment in Solid Tumors, J. of the National Cancer Institute 92(3):205-207 (2000), which is hereby incorporated by reference in its entirety. For example, the outcome may be survival (including overall survival or the duration of survival), progression-free interval, or survival after recurrence. The timing or duration of such events may be determined from about the time of diagnosis or from about the time treatment (e.g., chemotherapy) is initiated. Alternatively, the outcome may be based upon a reduction in tumor size, tumor volume, or tumor metabolism, or based upon overall tumor burden, or based upon levels of serum markers especially where elevated in the disease state. The outcome in some embodiments may be characterized as a complete response, a partial response, stable disease, and progressive disease, as these terms are understood in the art.

In certain embodiments, the presence or absence of a gene signature is indicative of a pathological complete response upon treatment with a particular candidate agent or combination (as already described). A pathological complete response, e.g., as determined by a pathologist following examination of tissue (e.g., breast or nodes in the case of breast cancer) removed at the time of surgery, generally refers to an absence of histological evidence of invasive tumor cells in the surgical specimen.

The present invention may further comprise conducting chemoresponse testing with a panel of chemotherapeutic agents on cultured cells from a cancer patient, to thereby add additional predictive value. That is, the presence of one or more gene expression signatures in tumor cells, and the in vitro chemoresponse results for the tumor specimen, are used to predict an outcome of treatment (e.g., survival, pCR, etc.). For example, where the gene expression profile and chemoresponse test both indicate that a tumor is sensitive or resistant to a particular treatment, the predictive value of the method may be particularly high. Chemoresponse testing may be performed via the CHEMOFX test, as described herein and as known in the art.

### EXAMPLES

### Materials and Methods

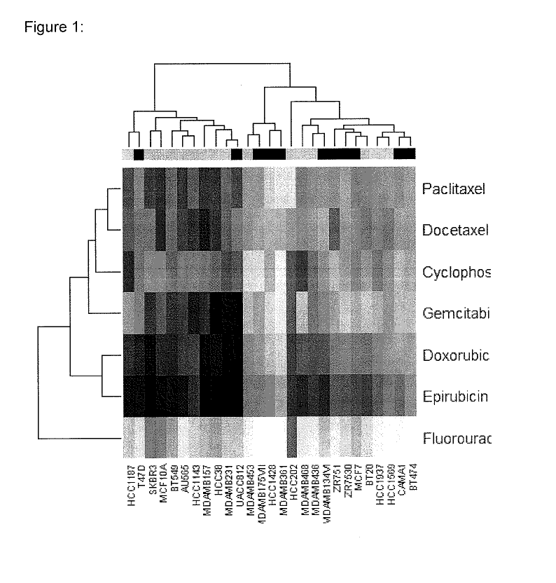

In this study, 27 breast cancer cell lines (as shown in FIG. 1) were obtained from American Type Culture Collection, Manassas, Va., USA. Cells were cultured in RPMI 1640 (Mediatech, Herndon, Va., USA). FBS was purchased from HyClone (Logan, Utah, USA). The following chemotherapeutic agents were used in the current study and prepared as recommended by the manufacturer in the growth media used for cell growth: paclitaxel, docetaxel, gemcitabine, cyclophosphamide, fluorouracil, doxorubicin, and epirubicin.

The CHEMOFX assay was performed as described previously (Mi, Holmes et al. 2008). Briefly, cells were treated with chemotherapeutic agents (untreated cells were used as a control). For each chemotherapeutic agent, ten serially diluted drug concentrations were tested in triplicate. After an incubation period of 72 hours, the cells were fixed, stained, and counted. The number of cells remaining after drug treatment was used to determine survival fraction (SF=average cell count dose x/average cell count control). Dose-response curves were plotted to determine the chemosensitivity, which is based on area under the curve (AUC) [(Mi, Holmes et al. 2008)]. Lower drug response scores indicate greater sensitivity. Lower drug response scores indicate greater sensitivity. The cells with the one third lowest values of AUC were deemed “sensitive,” while the cells with the highest one third values of AUC were deemed to be “resistant.”

Two dimensional hierarchical clustering was applied to the chemosensitivity data resulting from the CHEMOFX assay analyses. Cells that showed similar patterns of sensitivities to the drugs tested were grouped together. Likewise, drugs that showed similar response patterns among the cell lines tested were grouped together. For example, the two taxanes (paclitaxel and docetaxel) and the two anthracycline antitumor antibiotics (epirubicin and doxorubilcin) were each clustered together.

Raw gene expression data were downloaded from ArrayExpress, a public Affymetrix IDs were mapped to gene symbols. If a gene symbol was associated with multiple Affymetrix IDs, the one with the maximum IQR was chosen. Bioconductor software was used to apply non-specific gene filtering to these data sets. Briefly, the program filters the data as follows: suppose x denotes the expression value of gene i, then genes that do not satisfy the following two conditions are filtered out: 1) IQR(x)<0.5; 2) median(x) <log2(100).

To analyze how gene expression is related to multidrug response in breast cell lines, ER positive breast cells or ER negative breast cells, meta-analysis on breast cell lines, ER positive breast cell lines, and ER negative breast cell lines was conducted separately.

The algorithms used to perform the meta-analysis are as follows. Suppose there are a total of G genes and K studies (K=7 for this case). Let x_{gsk} denote the gene expression value of gene g, cell line s for drug k, s, 1≦g≦G, 1≦s≦S, 1≦k≦K. Let yₛₖ denote the AUC value for the cell line s for drug k. The regression coefficient β_{1gk} for gene g for drug k was computed using a standard linear regression model yₛₖ=β_{0Gk}+β_{1gk}x_{gsk}+∈_{sgk}, where ε_{sgk} is the normal error. Let t_{gk}=β_{1gk}/s_{gk}, where s_{gk} is the standard deviation of β_{1gk}s_{gk} is the standard deviation of β_{1gk}.

For each drug, the p-value of each gene was calculated by the following steps:
a. Compute the t_{gk} for gene g and drug k.
b. Permute the cell line's labels for B times, and similarly calculate the permuted statistics, t_{gk}^{(b)}, where 1≦g≦G, 1≦k≦K, 1≦b≦B.
c. Estimate the p-value of t_{gk} as

$${p}_{gk} = \frac{{\sum }_{b = 1}^{B} {\sum }_{{g}^{′} = 1}^{G} I ( \vert {t}_{{g}^{′} k}^{( b )} \vert \geq \vert {t}_{gk} \vert )}{B \cdot G}$$

and similarly calculate

$${p}_{gk}^{( b )} = \frac{{\sum }_{{b}^{′} = 1}^{B} {\sum }_{{g}^{′} = 1}^{G} I ( \vert {t}_{{g}^{′} k}^{( {b}^{′} )} \vert \geq \vert {t}_{gk}^{( b )} \vert )}{B \cdot G} .$$

d. Estimate π₀(k), the proportion of non-DE genes, as

$${\hat{π}}_{0} ( k ) = \frac{{\sum }_{g = 1}^{G} I ( {p}_{gk} ∈ A )}{G \cdot l ( A )} [ 1 ] .$$

We chose A=[0.5, 1] and thus l(A)=0.5.
e. Estimate the q-value of t_{gk} as

$${q}_{gk} = \frac{{\hat{π}}_{0} ( k ) \cdot {\sum }_{b = 1}^{B} {\sum }_{{g}^{′} = 1}^{G} I ( \vert {t}_{{g}^{′} k}^{( b )} \vert \geq \vert {t}_{gk} \vert )}{B \cdot {\sum }_{{g}^{′} = 1}^{G} I ( \vert {t}_{{g}^{′} k} \vert \geq \vert {t}_{gk} \vert )} .$$

The following steps are meta-analysis procedures to identify multi-drug response genes:
a. The rth rand statistic is used for meta-analysis: V_{g}=p_{g(5)}. Define V_{g}^{(b)}=p_{g(5)}^{(b)}.
b. Estimate the p-value of the genes in meta-analysis as

$$p ( {V}_{g} ) = \frac{{\sum }_{b = 1}^{B} {\sum }_{{g}^{′} = 1}^{G} I ( {V}_{{g}^{′}}^{( b )} \leq {V}_{g} )}{B \cdot G} .$$

c. Estimate π₀, the proportion of non-DE genes in the meta-analysis, as

$${\hat{π}}_{0} = \frac{{\sum }_{g = 1}^{G} I ( p ( {V}_{g} ) ∈ A )}{G \cdot l ( A )} .$$

We chose A=[0.5, 1] and thus l(A)=0.5.
d. Estimate the q-value in the meta-analysis as

$$q ( {V}_{g} ) = \frac{{\hat{π}}_{0} \cdot {\sum }_{b = 1}^{B} {\sum }_{{g}^{′} = 1}^{G} I ( {V}_{{g}^{′}}^{( b )} \leq {V}_{g} )}{B \cdot {\sum }_{{g}^{′} = 1}^{G} I ( {V}_{{g}^{′}} \leq {V}_{g} )} .$$

DE genes detected by the meta-analysis are denoted as Gₘₑₜₐ={g:q(V_{g})≦0.01}. These DE genes are considered as multi-drug response genes in this study.
Finally, multi-drug response genes were defined as those genes that were associated with resistance to at least 5 different drugs. We then referred to the Molecular Signature Database to evaluate the reported biological function of these genes.
Fold change values between sensitive and resistant cell lines were calculated by sorting the cell lines based on their AUC values. The top ⅓ of the cell lines are defined as sensitive and the bottom ⅓ of the cell lines are defined as resistant. For the fold change the calculation is done as follows for each gene: mean raw expression value for the drug sensitive group/mean raw expression value for the drug resistant group.

### Results

ChemoFx analysis was performed on 27 well-characterized breast cancer cell lines to measure their response to the following 7 widely used chemotherapeutic agents: paclitaxel, docetaxel, gemcitabine, cyclophosphamide, fluorouracil, doxorubicin, and epirubicin (FIG. 1).

As with primary tumors, these cell lines exhibited a heterogeneous response to the drugs (FIG. 2). Generally speaking, three clusters of cell lines were identified based on their responses to the different agents resulted. The first cluster consisted of 9 cell lines that were pan-resistant to the tested drugs. This cluster was enriched (⅞) in estrogen receptor (ER) positive cells. The second cluster included 8 cell lines that were pan-sensitive to the tested drugs. All of them were ER negative. The third cluster consisted of 11 cell lines that showed a heterogeneous response to the tested drugs and were both ER positive (4) and ER negative (7).

We also performed hierarchical clustering on the 7 chemotherapeutic drugs based on the drug response patterns of the cells. We found that paclitaxel and docetaxel were clustered together as were doxrubicin and epirubicin.

Through pharmacogenomic analysis, 524 genes were identified to be related to multidrug response for all breast cell lines. Many of these genes are related to ER status. In ER negative breast cancer cell lines, 32 genes were identified to be related to multidrug response, and 21 of them are in the list of 524 genes. In ER positive cell lines, 188 genes were related to multidrug response, and 70 of them exist in the list of 524 genes. Only 3 gene was related to multidrug response for both ER positive breast cell lines and ER negative breast cell lines (DBI, TOP2A, PMVK).

### Discussion

A pharmacogenomic analysis of 27 breast cancer cell lines identified 32 genes related to multidrug response in ER negative breast cells, and 188 genes related to multidrug response in ER positive cell lines.

A functional analysis of these genes indicates that they are related to several diverse biological functions, supporting the current understanding that multidrug response is the result of multiple mechanisms.

A key issue of using cell lines is how good the surrogate can proximate patient outcome. To date, various CSRA analysis have been used, including MTT and ATP. In this example, we applied CHEMOFX. Specifically, 7 classical drugs were tested in a collection of 27 breast cell lines. The drug response pattern of cell lines also suggests that CHEMOFX is a good proxy through several aspects. First, these cell lines show (exhibit) heterogeneous responses, similar to clinical observations. In addition, these cell lines show that ER status strongly correlates with drug responses for most chemotherapy drugs—ER positive cell lines tend to be more resistant, while ER negative cell lines tend to be sensitive. This is consistent with previous publications. Furthermore, our drug clustering also supports the accuracy of CHEMOFX. The 7 drugs tested represent the major classes of anticancer drugs. Cyclophosphamide is an alkylating agent. Doxorubicin and Epirubicin are antibiotics. Paclitaxel and Docetaxel are taxanes, and “5-Fu” is an antimetabolite that acts as a thymidylate synthase inhibitor. Thus, Paclitaxel and Docetaxel were clustered together, and Epirubicin and Doxrubicin were clustered together.

The analysis disclosed herein is useful for understanding multidrug resistance to cytotoxic chemotherapy drugs, and to individualize patient treatment.

For instance, where a multidrug resistant signature is present, a physician might consider some less conventional chemotherapeutic treatments (e.g., not represented by the agents disclosed herein), or might consider more aggressive radiation of surgical intervention.

Further, genes associated with multiple drug resistance have helped determine how some cancers can be resistant to drug treatment and have provided potential targets. Similarly, analysis of oncogenes has demonstrated that such genes have wild-type counterparts often involved in signal transduction for growth control pathways. Many of these genes code for proteins that are regulated by tyrosine kinases, making phospho-tyrosine a popular target for drug development.

### REFERENCES

The following references are hereby incorporated by reference in their entirety.

Chang, J. C., E. C. Wooten, et al. (2003). “Gene expression profiling for the prediction of therapeutic response to docetaxel in patients with breast cancer.” Lancet 362(9381): 362-369.

Dan, S., T. Tsunoda, et al. (2002). “An integrated database of chemosensitivity to 55 anticancer drugs and gene expression profiles of 39 human cancer cell lines.” Cancer Res 62(4): 1139-1147.

Gianni, L., M. Zambetti, et al. (2005). “Gene expression profiles in paraffin-embedded core biopsy tissue predict response to chemotherapy in women with locally advanced breast cancer.” J Clin Oncol 23(29): 7265-7277.

Gyorffy, B., P. Surowiak, et al. (2006). “Gene expression profiling of 30 cancer cell lines predicts resistance towards 11 anticancer drugs at clinically achieved concentrations.” Int J Cancer 118(7): 1699-1712.

Hess, K. R., K. Anderson, et al. (2006). “Pharmacogenomic predictor of sensitivity to preoperative chemotherapy with paclitaxel and fluorouracil, doxorubicin, and cyclophosphamide in breast cancer.” J Clin Oncol 24(26): 4236-4244.

Iwao-Koizumi, K., R. Matoba, et al. (2005). “Prediction of docetaxel response in human breast cancer by gene expression profiling.” J Clin Oncol 23(3): 422-431.

Kang, H. C., I. J. Kim, et al. (2004). “Identification of genes with differential expression in acquired drug-resistant gastric cancer cells using high-density oligonucleotide microarrays.” Clin Cancer Res 10(1 Pt 1): 272-284.

Liedtke, C., C. Hatzis, et al. (2009). “Genomic grade index is associated with response to chemotherapy in patients with breast cancer.” J Clin Oncol 27(19): 3185-3191.

Mariadason, J. M., D. Arango, et al. (2003). “Gene expression profiling-based prediction of response of colon carcinoma cells to 5-fluorouracil and camptothecin.” Cancer Res 63(24): 8791-8812.

Mi, Z., F. A. Holmes, et al. (2008). “Feasibility assessment of a chemoresponse assay to predict pathologic response in neoadjuvant chemotherapy for breast cancer patients.” Anticancer Res 28(3B): 1733-1740.

O'Driscoll, L. and M. Clynes (2006). “Molecular markers of multiple drug resistance in breast cancer.” Chemotherapy 52(3): 125-129.

Paik, S., G. Tang, et al. (2006). “Gene expression and benefit of chemotherapy in women with node-negative, estrogen receptor-positive breast cancer.” J Clin Oncol 24(23): 3726-3734.

Staunton, J. E., D. K. Slonim, et al. (2001). “Chemosensitivity prediction by transcriptional profiling.” Proc Natl Acad Sci USA 98(19): 10787-10792.

van de Vijver, M. J., Y. D. He, et al. (2002). “A gene-expression signature as a predictor of survival in breast cancer.” N Engl J Med 347(25): 1999-2009.

Wang, Y., J. G. Klijn, et al. (2005). “Gene-expression profiles to predict distant metastasis of lymph-node-negative primary breast cancer.” Lancet 365(9460): 671-679.

1. A method for preparing a gene expression profile of a breast cancer cell, tumor, or cell line, the profile being indicative of drug response, comprising, extracting RNA from a breast tumor sample or cell culture derived therefrom, and determining the gene expression level of genes listed in one or more of FIGS. 3, 4, and/or 5.

2. The method of claim 1, wherein the breast tumor or cell line is ER positive, and the gene expression profile contains the level of expression for a plurality of genes listed in FIG. 3.

3. The method of claim 1, wherein the breast tumor or cell line is ER negative, and the gene expression profile contains the level of expression for a plurality of genes listed in FIG. 4.

4. The method of claim 1, wherein the gene expression profile is indicative of response to at least two agents selected from a Taxol; an antibiotic; an antimetabolite; and an alkylating agent.

5. The method of claim 4, wherein the Taxol is docetaxel or placlitaxel.

6. The method of claim 4, wherein the antimetabolite is 5-fluorouracil or gemcitabine.

7. The method of claim 4, wherein the alkylating agent is cyclophosphamide.

8. The method of claim 4, wherein the antibiotic is doxorubicin or epirubicin.

9. The method of claim 1, further comprising, determining the presence of one or more gene expression signatures indicative of multidrug response.

10. The method of claim 1, further comprising conducting a Chemoresponse assay.

11. A method for preparing a gene expression profile of a breast tumor, comprising,
determining an estrogen receptor (ER) status for the tumor;
extracting RNA from the tumor sample or cell culture derived therefrom; and
determining a gene expression profile for the tumor, where the gene expression profile includes the level of expression for a plurality genes listed in one or more of FIGS. 3, 4, and/or 5.

12. The method of claim 11, wherein the breast tumor or cell line is ER positive, and the gene expression profile contains the level of expression for a plurality of genes listed in FIG. 3.

13. The method of claim 11, wherein the breast tumor or cell line is ER negative, and the gene expression profile contains the level of expression for a plurality of genes listed in FIG. 4.

14. The method of claim 11, wherein the gene expression profile is indicative of resistance to at least two agents selected from a Taxol; an antibiotic; an antimetabolite; and an alkylating agent.

15. The method of claim 14, wherein the Taxol is docetaxel or placlitaxel.

16. The method of claim 14, wherein the antimetabolite is 5-fluorouracil or gemcitabine.

17. The method of claim 14, wherein the alkylating agent is cyclophosphamide.

18. The method of claim 14, wherein the antibiotic is doxorubicin or epirubicin.

19. The method of claim 11, further comprising, determining the presence of one or more gene expression signatures indicative of multidrug resistance.

20. The method of claim 11, further comprising conducting a Chemoresponse assay.
